(19) United States
(12) Patent Application Publication
(10) Pub. No.: US 2016/0113912 A1
(43) Pub. Date: Apr. 28, 2016

(54) AN IMPROVED PROCESS FOR THE PREPARATION OF APIXABAN AND INTERMEDIATES THEREOF

(71) Applicant: CADILA HEALTHCARE LIMITED, Gujarat (IN)

(72) Inventors: Shriprakash Dhar DWIVEDI, Gujarat (IN); Kumar Kamlesh SINGH, Gujarat (IN); Nitin TANDON, Gujarat (IN); Digambar WARE, Gujarat (IN)

(21) Appl. No.: 14/898,089

(22) PCT Filed: Jun. 17, 2014
(86) PCT No.: PCT/IN2014/000401

§ 371 Date: Dec. 11, 2015

(30) Foreign Application Priority Data
Jun. 18, 2013 (IN) 2059/MUM/2013
Oct. 28, 2013 (IN) 3396/MUM/2013

(51) Int. Cl. A61K 31/437

(52) U.S. Cl. CPC A61K 31/437

(57) ABSTRACT

The present invention relates to an improved process for the preparation of apixaban and intermediates thereof. In particular, the invention relates to an improved process for the preparation of an amorphous form of apixaban. The invention also relates to a pharmaceutical composition comprising an amorphous form of apixaban for oral administration as an antithrombotic agent.

### FIELD OF THE INVENTION

The present invention relates to an improved process for the preparation of apixaban and intermediates thereof. In particular, the invention relates to an improved process for the preparation of an amorphous form of apixaban. The invention also relates to a pharmaceutical composition comprising an amorphous form of apixaban for oral administration as an antithrombotic agent.

### BACKGROUND OF THE INVENTION

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

“Apixaban” is chemically known as 4,5,6,7-tetrahydro-1-(4-methoxyphenyl)-7-oxo-6-[4-(2-oxo-1-piperidinyl)phenyl]-1H-pyrazolo[3,4-c]pyridine-3-carboxamide (CAS name) or 1-(4-methoxyphenyl)-7-oxo-6-[4-(2-oxo-1-piperidinyl)phenyl]-4,5,6,7-tetrahydro-1H-pyrazolo[3,4-c]pyridine-3-carboxamide (IUPAC name) of Formula (I).

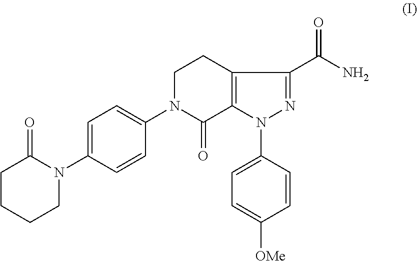 [SMILES: COc1ccc(-n2nc(C(N)=O)c3c2C(=O)N(c2ccc(N4CCCCC4=O)cc2)CC3)cc1]

International (PCT) publication No. WO 2003/026652 A1 (the WO '652 A1) discloses the process for the preparation of pyrazole-pyridine derivatives. U.S. Pat. No. 6,967,208, the family equivalent of WO '652 A1 discloses apixaban, has utility as a factor Xa inhibitor, and is developed for oral administration in a variety of indications that require the use of an antithrombotic agent.

U.S. Pat. Nos. 7,005,435 B2, 6,989,391 B2, 6,995,172 B2, 7,338,963 B2, 7,371,761 B2, 7,531,535 B2, 7,691,846 B2 and 7,960,411 B2 disclose various analogues compounds of apixaban. All the patents are incorporated herein by reference in their entirety.

International (PCT) publication No. WO 2003/049681 A2 and its corresponding U.S. Pat. No. 6,919,451 B2 and U.S. Pat. No. 7,153,960 B2 disclose process for the preparation of apixaban and other pyrazole-pyridine derivatives.

International (PCT) publication No. WO 2007/001385 A2 and its corresponding U.S. Pat. No. 7,396,932 B2 (the U.S. Pat. No. '932 B2) discloses the process for the preparation of pyrazole-pyridine derivatives as depicted in scheme-1. The U.S. Pat. No. '932 B2 also disclose crystalline form N-1 and form H2-2 of apixaban along with the unit cell data thereof.

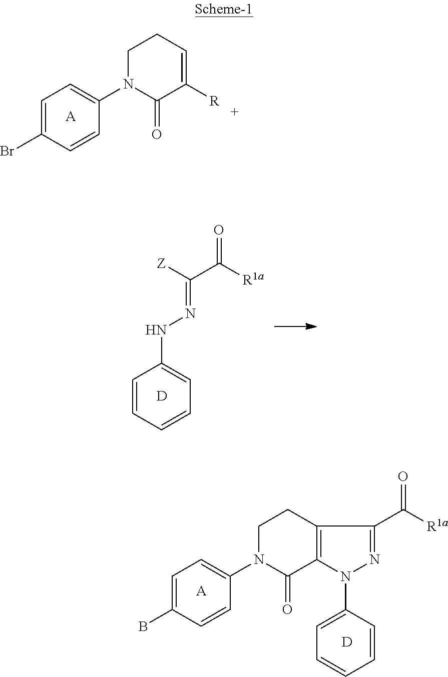

wherein Z is selected from Cl, Br, I, OSO₂Me, OSO₂Ph, and OSO₂Ph-p-Me;
ring D is selected from phenyl, 2-fluorophenyl, 3-chlorophenyl, and 4-methoxyphenyl;
R^{1a} is selected from CH₃, CH₂CH₃, CH₂CH₂CH₃, OCH₃, OCH₂CH₃, OCH₂CH₂CH₃, OCH(CH₃)₂, OCH₂CH₂CH₂CH₃, OCH(CH₃)CH₂CH₃, OCH₂CH(CH₃)₂, OC(CH₃)₃, O-phenyl, OCH₂-phenyl, OCH₂CH₂-phenyl, and OCH₂CH₂H₂-phenyl;
R is selected from Cl, Br, and I; ring A is substituted with 0-1R⁴; B is NO₂.

International (PCT) publication No. WO 2003/048081 A2 and WO 2003/048158 A1 discloses the process for the preparation of pyrazole-pyridine derivatives by reacting the 3-morpholino-1-(4-nitrophenyl)-5,6-dihydropyridin-2(1H)-one and (Z)-ethyl 2-chloro-2-(2-(4-chlorophenyl)hydrazono)acetate to obtain pyrazole-pyridine derivative as depicted in scheme-2.

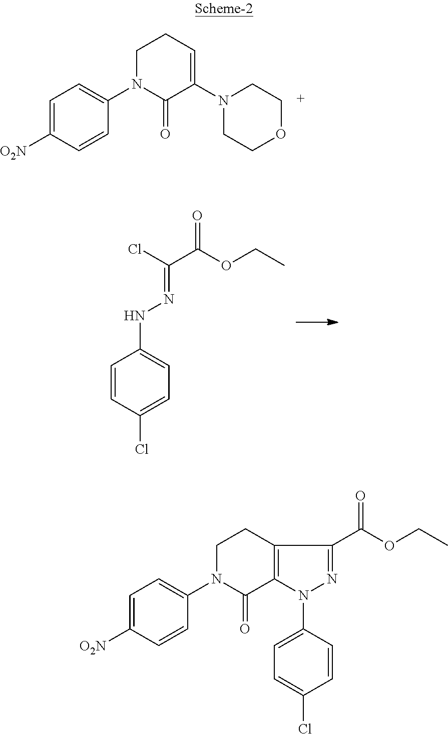

Journal of Labelled Compounds and Radiopharmaceuticals Vol. 54 (8) Pg. 418-425 (2011) discloses a nine-step synthesis for the preparation of [¹⁴C]apixaban with the label in the central lactam ring and three-step synthesis for the preparation of [¹⁴C]apixaban with the label in the outer lactam ring starting from 4-nitroaniline.

IP.com Journal Vol. 12(11A) Pg. 10, (2012) discloses the synthesis of apixaban by reduction of nitro group of ethyl 1-(4-methoxyphenyl)-6-(4-nitrophenyl)-7-oxo-4,5,6,7-tetrahydro-1H-pyrazolo[3,4-c]pyridine-3-carboxylate by usage of 10% Pd/C catalyst in presence of formic acid and potassium formate and amidation with ethylene glycol saturated with ammonia to obtain 6-(4-aminophenyl)-1-(4-methoxyphenyl)-7-oxo-4,5,6,7-tetrahydro-1H-pyrazolo[3,4-c]pyridine-3-carboxamide followed by N-acylation with 5-bromovaleroyl chloride and intramolecular heterocyclization of the intermediate 6-(4-(5-bromopentanamido)phenyl)-1-(4-methoxyphenyl)-7-oxo-4,5,6,7-tetrahydro-1H-pyrazolo[3,4-c]pyridine-3-carboxamide.

IP.com Journal Vol. 12(12A) Pg. 21 (2012) discloses the preparation of apixaban precursor 6-(4-aminophenyl)-1-(4-methoxyphenyl)-7-oxo-4,5,6,7-tetrahydro-1H-pyrazolo[3,4-c]pyridine-3-carboxamide by treatment of ethyl 6-(4-aminophenyl)-1-(4-methoxyphenyl)-7-oxo-4,5,6,7-tetrahydro-1H-pyrazolo[3,4-c]pyridine-3-carboxylate with ammonia. The crystalline forms of intermediates are also reported and characterized by the powder x-ray diffraction analysis.

CN 102675314 A discloses the process for the preparation of apixaban by cyclization of p-nitroaniline with 5-chloro-pentanoyl chloride or 5-bromo-pentanoyl chloride; the resulting 1-(4-nitrophenyl)-2-piperidinone underwent dichlorination with phosphorus pentachloride followed elimination; the resulting 3-chloro-5,6-dihydro-1-(4-nitrophenyl)-2(1H)-pyridinone underwent reaction with ethyl (2Z)-chloro[(4-methoxyphenyl)hydrazono]acetate; the resulting ethyl 4,5,6,7-tetrahydro-1-(4-methoxyphenyl)-6-(4-nitrophenyl)-7-oxo-1H-pyrazolo [3,4-c]pyridine-3-carboxylate underwent reduction followed by cyclization with 5-chlorovalaroyl chloride or 5-bromovalaroyl chloride; the resulting intermediate underwent amidation to give apixaban.

Journal of Medicinal Chemistry (2007), 50(22), 5339-5356 discloses the process for the preparation of apixaban and other derivatives. The reaction scheme-7 in the reference article discloses the preparation of compound 47a which is outlined herein scheme-3.

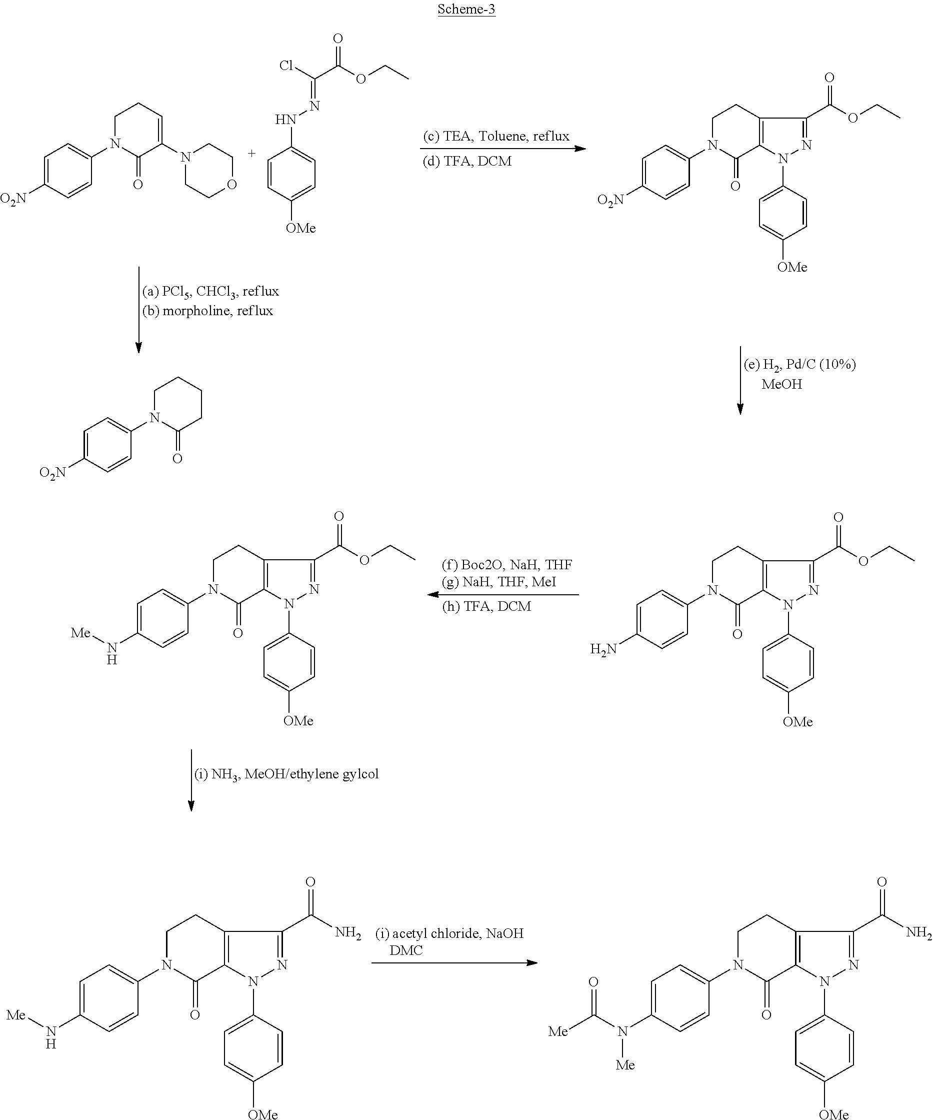

U.S. Patent Application Publication No. 2007/0203178 A1 discloses crystalline solvates of apixaban viz. dimethyl formamide solvate DMF-5 and formamide solvate Form FA-2 of apixaban characterized by unit cell parameters.

WO 2011/0106478 A2 discloses a composition comprising crystalline apixaban particles having a mean particle size equal to or less than about 89 μm and a pharmaceutically acceptable diluent or carrier.

WO 2012/0168364 A1 discloses a process for the preparation of apixaban via novel intermediate and crystalline form a of apixaban which is designated as sesquihydrate having water content between about 4.5 and 6.5%. The crystalline form a of apixaban is characterized by x-ray powder diffraction and differential scanning calorimetry.

WO 2013/119328 A1 discloses crystalline Form-I, Form-II and Form-III of apixaban.

WO 2013/164839 A2 discloses amorphous form of apixaban and process for preparation and composition thereof.

U.S. Pub. No. 2013/0245267 A1 discloses amorphous form of apixaban and process for its preparation.

WO 2014/056434 A1 discloses crystalline form and amorphous form of apixaban.

Polymorphism is the occurrence of different crystalline forms of a single compound and it is a property of some compounds and complexes. Thus, polymorphs are distinct solids sharing the same molecular formula, yet each polymorph may have distinct physical properties. Therefore, a single compound may give rise to a variety of polymorphic forms where each form has different and distinct physical properties, such as different solubility profiles, different melting point temperatures and/or different x-ray diffraction peaks. Since the solubility of each polymorph may vary, identifying the existence of pharmaceutical polymorphs is essential for providing pharmaceuticals with predicable solubility profiles. It is desirable to investigate all solid-state forms of a drug, including all polymorphic forms, and to determine the stability, dissolution and flow properties of each polymorphic form. The polymorphic forms of a compound can be distinguished in a laboratory by X-ray diffraction spectroscopy and by other methods such as, infrared spectrometry. For a general review of polymorphs and the pharmaceutical applications of polymorphs, See G. M. Wall, Pharm Manuf. 3, 33 (1986); J. K. Haleblian and W. McCrone, J. Pharm. Sci., 58, 911 (1969); and J. K. Haleblian, J. Pharm. Sci., 64, 1269 (1975), all of which are incorporated herein by reference.

In view of the above, discovering new polymorphic forms and solvates of a pharmaceutical product can provide materials having desirable processing properties, such as ease of handling, ease of processing, storage stability, and ease of purification or as desirable intermediate crystal forms that facilitate conversion to other polymorphic forms. New polymorphic forms and solvates of a pharmaceutically useful compound thereof can also provide opportunities to improve the performance characteristics of a pharmaceutical product. They can also enlarge the repertoire of materials available to a formulation scientist for formulation optimization, for example by providing a product with different properties, e.g., better processing or handling characteristics, improved dissolution profile, or improved shelf-life. For at least these reasons, there is a need for additional polymorphs of apixaban.

The reported processes herein involve complex synthesis which is expensive and danger of reagents and the drastic reaction conditions are required. In the view of the above, it is therefore, desirable to provide an efficient, more economical, less hazardous and eco-friendly process for the preparation apixaban.

### SUMMARY OF THE INVENTION

In one embodiment, there is provided a crystalline form of apixaban of Formula (I)

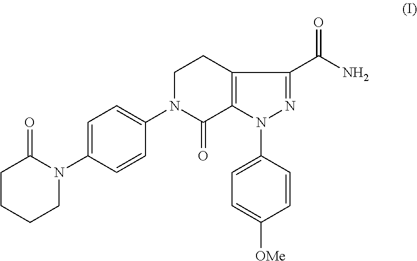 [SMILES: COc1ccc(-n2nc(C(N)=O)c3c2C(=O)N(c2ccc(N4CCCCC4=O)cc2)CC3)cc1]

In another embodiment, there is provided an intermediate of apixaban comprises compounds of Formula (IIA), (IIB) and (IVA).

In another embodiment, there is provided isolated compounds of Formula (IIA), (IIB) and (IVA).

In another embodiment, the isolated intermediates are compounds of Formula (II), Formula (IIA), Formula (IIB) and Formula (IVA).

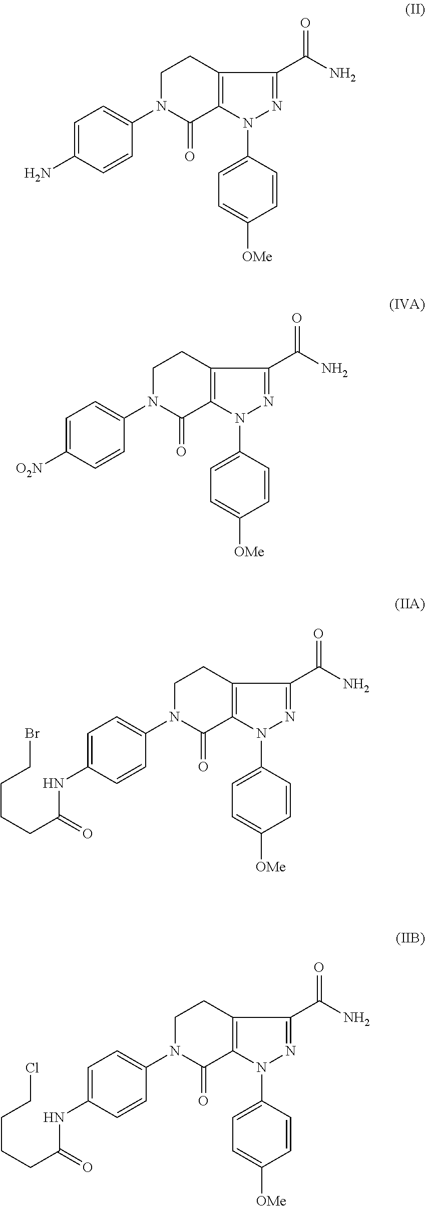 [SMILES: COc1ccc(-n2nc(C(N)=O)c3c2C(=O)N(c2ccc(N)cc2)CC3)cc1 ; COc1ccc(-n2nc(C(N)=O)c3c2C(=O)N(c2ccc([N+](=O)[O-])cc2)CC3)cc1 ; COc1ccc(-n2nc(C(N)=O)c3c2C(=O)N(c2ccc(NC(=O)CCCCBr)cc2)CC3)cc1 ; COc1ccc(-n2nc(C(N)=O)c3c2C(=O)N(c2ccc(NC(=O)CCCCCl)cc2)CC3)cc1]

In another embodiment, there is provided an improved process for the preparation of apixaban of Formula (I)

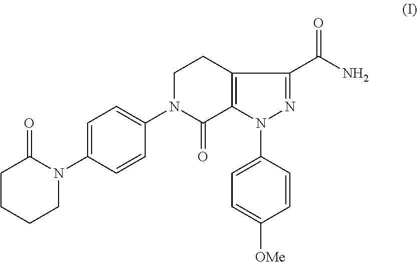 [SMILES: COc1ccc(-n2nc(C(N)=O)c3c2C(=O)N(c2ccc(N4CCCCC4=O)cc2)CC3)cc1]

the process comprising:
(a) reacting (Z)-ethyl 2-chloro-2-(2-(4-methoxyphenyl)hydrazono)acetate of Formula (V) with 3-morpholino-1-(4-nitrophenyl)-5,6-dihydropyridin-2(1H)-one of Formula (VI) in one or more organic solvents in the presence of a base to obtain compound (IV);

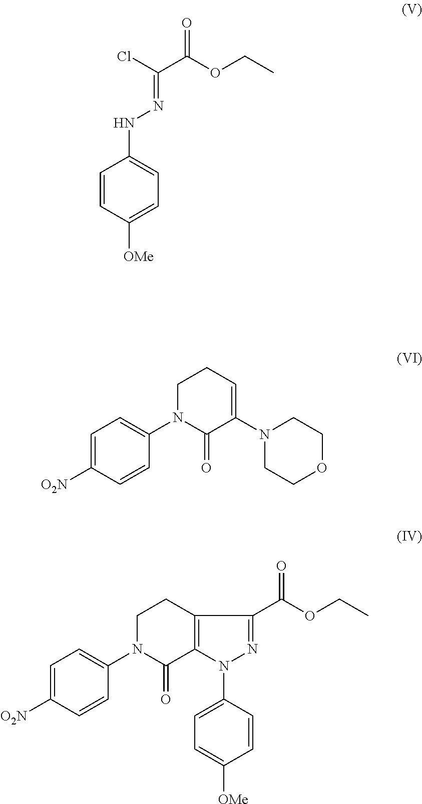 [SMILES: CCOC(=O)/C(Cl)=N/Nc1ccc(OC)cc1 ; O=C1C(N2CCOCC2)=CCCN1c1ccc([N+](=O)[O-])cc1 ; CCOC(=O)c1nn(-c2ccc(OC)cc2)c2c1CCN(c1ccc([N+](=O)[O-])cc1)C2=O]

(b) reducing the compound (IV) with a reducing agent to obtain compound (III);

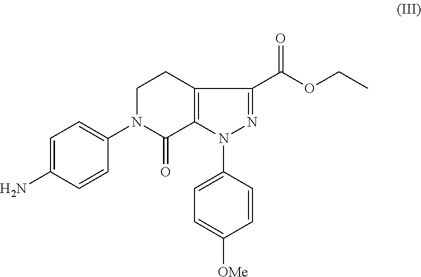 [SMILES: CCOC(=O)c1nn(-c2ccc(OC)cc2)c2c1CCN(c1ccc(N)cc1)C2=O]

(c) amidating the compound (III) with an amidating source in one or more organic solvents to obtain compound (II);

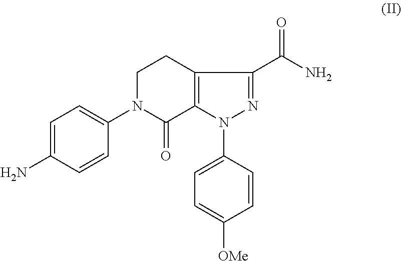 [SMILES: COc1ccc(-n2nc(C(N)=O)c3c2C(=O)N(c2ccc(N)cc2)CC3)cc1]

(d) reacting the compound (II) with 5-chlorovalaroyl chloride in the presence of a base to obtain compound (IIB);

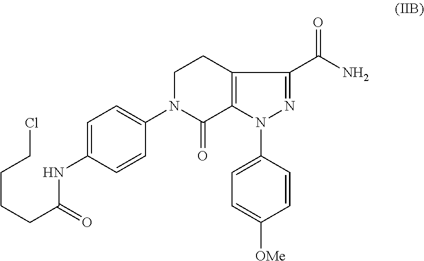 [SMILES: COc1ccc(-n2nc(C(N)=O)c3c2C(=O)N(c2ccc(NC(=O)CCCCCl)cc2)CC3)cc1]

(e) cyclizing of compound (IIB) in the presence of a base in one or more organic solvents to obtain apixaban of Formula (I); and
(f) optionally, converting apixaban of Formula (I) to an amorphous form, provided that the compound (IIB) is isolated as crystalline solid.

In another embodiment, 5-chlorovalaroyl chloride in step (d) may be replaced by 5-bromovalaroyl chloride to obtain compound of Formula (IIA).

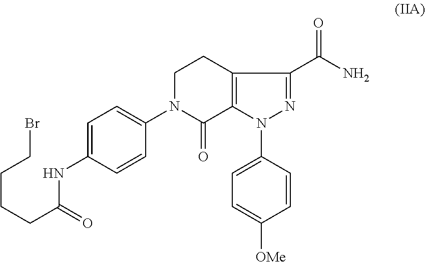 [SMILES: COc1ccc(-n2nc(C(N)=O)c3c2C(=O)N(c2ccc(NC(=O)CCCCBr)cc2)CC3)cc1]

In another embodiment, there is provided an improved process for the preparation of apixaban of Formula (I)

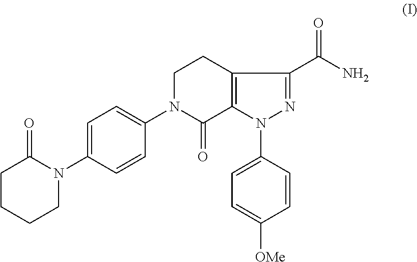 [SMILES: COc1ccc(-n2nc(C(N)=O)c3c2C(=O)N(c2ccc(N4CCCCC4=O)cc2)CC3)cc1]

the process comprising:
(a) reacting (Z)-ethyl 2-chloro-2-(2-(4-methoxyphenyl)hydrazono)acetate of Formula (V) with 3-morpholino-1-(4-nitrophenyl)-5,6-dihydropyridin-2 (1H)-one of Formula (VI) in one or more organic solvents in the presence of a base to obtain compound (IV);

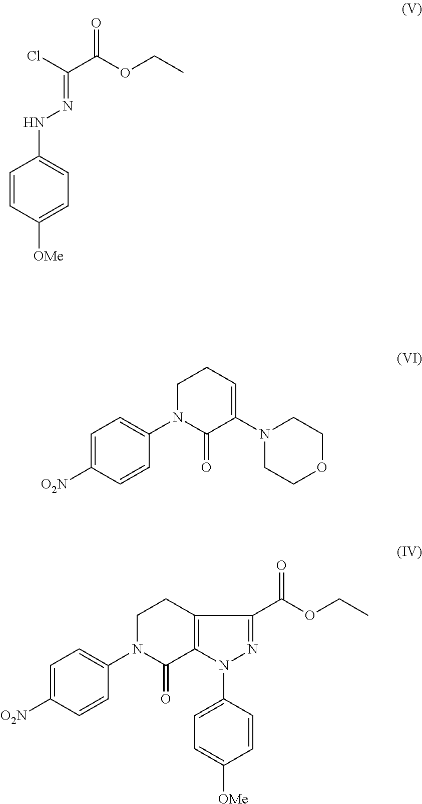 [SMILES: CCOC(=O)/C(Cl)=N/Nc1ccc(OC)cc1 ; O=C1C(N2CCOCC2)=CCCN1c1ccc([N+](=O)[O-])cc1 ; CCOC(=O)c1nn(-c2ccc(OC)cc2)c2c1CCN(c1ccc([N+](=O)[O-])cc1)C2=O]

(b) amidating the compound (IV) with an amidating source in one or more organic solvents to obtain compound (IVA);

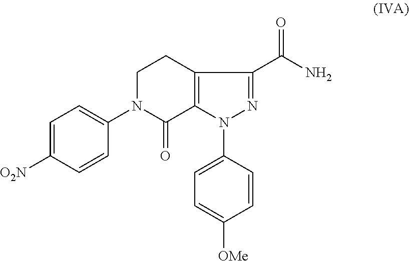 [SMILES: COc1ccc(-n2nc(C(N)=O)c3c2C(=O)N(c2ccc([N+](=O)[O-])cc2)CC3)cc1]

(c) reducing the compound (IVA) with a reducing agent to obtain compound (II);

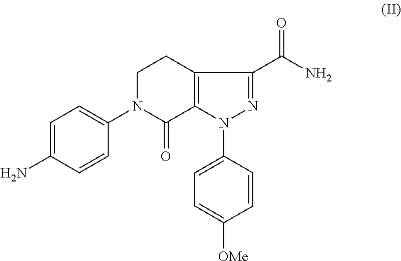 [SMILES: COc1ccc(-n2nc(C(N)=O)c3c2C(=O)N(c2ccc(N)cc2)CC3)cc1]

(d) reacting the compound (II) with 5-chlorovalaroyl chloride in the presence of a base to obtain compound (IIB);

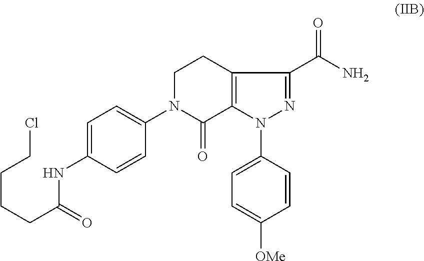 [SMILES: COc1ccc(-n2nc(C(N)=O)c3c2C(=O)N(c2ccc(NC(=O)CCCCCl)cc2)CC3)cc1]

(e) cyclizing the compound (IIB) in the presence of a base in one or more organic solvents to obtain apixaban of Formula (I); and
(f) optionally, converting apixaban of Formula (I) to an amorphous form, provided that the compound (IIB) is isolated as crystalline solid.

In another embodiment, there is provided polymorphic forms of isolated intermediates of Formula (II), Formula (IVA), Formula (IIA) and Formula (IIB).

In another embodiment, there is provided use of isolated intermediates of Formula (II), Formula (IVA), Formula (IIA) and Formula (IIB) in their polymorphic forms for the preparation of apixaban of Formula (I).

In another embodiment, there is provided a crystalline apixaban prepared by the process of the present invention having purity of at least about 99% by area percentage of HPLC.

In another embodiment, there is provided an amorphous apixaban prepared by the process of the present invention having purity of at least about 99% by area percentage of HPLC.

In another embodiment, there is provided crystalline apixaban prepared by the process of the present invention having a particle size distribution having (D₁₀) of about 50 μm or less, (D₅₀) of about 100 μm or less and (D₉₀) of about 150 μm or less. In a further embodiment, the apixaban may be micronized to achieve the better particle size distribution in order to make suitable Formulation.

In another embodiment, there is provided a pharmaceutical composition comprising crystalline apixaban together with one or more pharmaceutically acceptable excipients, diluents and carriers.

### BRIEF DESCRIPTION OF THE ACCOMPANYING DRAWINGS

FIG. 1: Illustrates XRPD of crystalline apixaban (I).

FIG. 2: Illustrates DSC of crystalline apixaban (I).

FIG. 3: Illustrates XRPD of crystalline form of compound (IV).

FIG. 4: Illustrates DSC of crystalline form of compound (IV).

FIG. 5: Illustrates TGA of crystalline form of compound (IV).

FIG. 6: Illustrates XRPD of crystalline form of compound (IVA).

FIG. 7: Illustrates DSC of crystalline form of compound (IVA).

FIG. 8: Illustrates TGA of crystalline form of compound (IVA).

FIG. 9: Illustrates XRPD of compound (III).

FIG. 10: Illustrates DSC of compound (III).

FIG. 11: Illustrates TGA of compound (III).

FIG. 12: Illustrates XRPD of crystalline form of compound (II).

FIG. 13: Illustrates DSC of crystalline compound (II).

FIG. 14: Illustrates TGA of crystalline compound (II).

FIG. 15: Illustrates XRPD of compound (IIA).

FIG. 16: Illustrates DSC of compound (IIA).

FIG. 17: Illustrates XRPD of crystalline form of compound (IIB).

FIG. 18: Illustrates DSC of crystalline compound (IIB).

FIG. 19: Illustrates XRPD of amorphous form of apixaban (I).

### DETAILED DESCRIPTION OF THE INVENTION

The above and other objects of the present invention are achieved by the process of the present invention, which leads to an improved process for the preparation of apixaban of Formula (I) and intermediates thereof.

Optionally, the solution, prior to any solids formation, can be filtered to remove any undissolved solids, solid impurities prior to removal of organic solvents. Any filtration system and filtration techniques known in the art can be used.

All ranges recited herein include the endpoints, including those that recite a range “between” two values. Terms such as “about”, “generally”, “substantially,” are to be construed as modifying a term or value such that it is not an absolute. This includes, at very least, the degree of expected experimental error, technique error and instrument error for a given technique used to measure a value.

As used here in the term “obtaining” includes filtration, filtration under vacuum, centrifugation, and decantation for isolation of the product. The product obtained may be further or additionally dried to achieve the desired moisture values.

For example, the product may be dried in a tray drier, dried under vacuum and/or in a Fluid Bed Drier. The product may be proceed for further reaction with or without isolation and with or without drying in case of the product was isolated.

As used herein the term “substantially pure” means a compound having a purity of at least about 98%, by area percentage of HPLC. In particular, the compound is having a purity of at least about 99%, more particularly, a purity of at least about 99.5%, further more particularly, a purity of at least about 99.8%, most particularly, a purity of at least about 99.9% by area percentage of HPLC.

As used herein the term “substantially amorphous” herein means amorphous compound having less than about 25% of crystalline compound. In particular, the amorphous compound having less than about 20%, more particularly less than about 15%, most particularly less than about 10% of crystalline compound.

As used herein the term “substantially crystalline” herein means crystalline compound having less than about 20% of amorphous compound. In particular, the crystalline compound having less than about 20%, more particularly less than about 15%, most particularly less than about 10% of amorphous compound.

As used herein, the term “stable apixaban” means the amorphous apixaban does not convert to any other solid form when stored at a temperature of up to about 40° C. and at a relative humidity of about 25% to about 75% for about three months or more.

As used herein, the term “solid dispersion” means any solid composition having at least two components. In certain embodiments, a solid dispersion as disclosed herein includes an active ingredient apixaban dispersed among at least one other component, for example a polymer.

The term “immobilize” as used herein with reference to the immobilization of the active compound i.e. apixaban in the polymer matrix, means that molecules of the active compound interact with molecules of the polymer in such a way that the molecules of the apixaban are held in the aforementioned matrix and prevented from crystal nucleation due to lack of mobility.

The terms herein below are interchangeable and used in the description.

“TEA” refers to triethylamine

“TBA” refers to tert-butyl amine

“DIPA” refers to diisopropyl amine

“DIPEA” refers to diisopropyl ethylamine

“DBU” refers to 1,8-diazabicyclo[5.4.0]undec-7-ene

“DABCO” refers to 1,4-diazabicyclo[2.2.2]octane

“DBN” refers to 1,5-diazabicyclo[4.3.0]non-5-ene

In one general aspect, there is provided a crystalline form of apixaban of Formula (I)

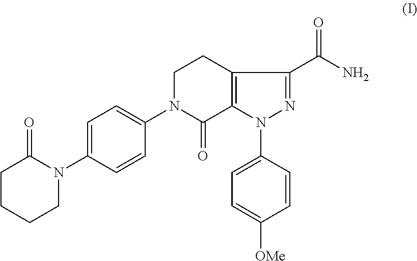 [SMILES: COc1ccc(-n2nc(C(N)=O)c3c2C(=O)N(c2ccc(N4CCCCC4=O)cc2)CC3)cc1]

In another general aspect, there is provided a crystalline form of apixaban (I) characterized by X-ray powder diffraction pattern having characteristic peaks expressed in degrees 2θ (±0.2° 20) at 5.9°, 6.9°, 13.4°, 14.9°, 16.0°, 17.3°, 21.4°, 22.5°, 24.2°, and 25.8°±0.2 2θ.

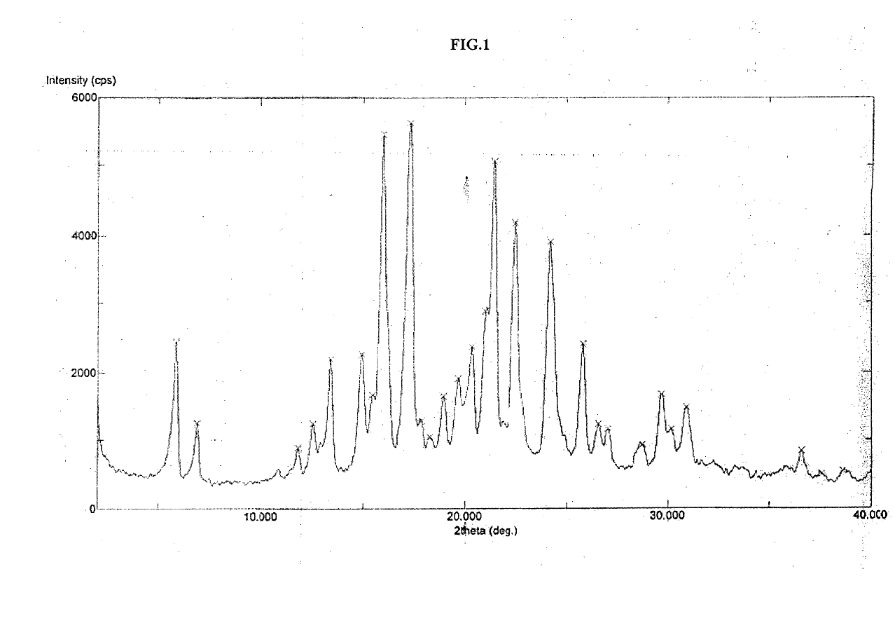

In general, the crystalline form of apixaban of Formula (I) is further characterized by X-ray powder diffraction pattern having characteristic peaks expressed in degrees 2θ (±0.2° 2θ) at 5.9°, 6.9°, 12.6°, 13.4°, 14.9°, 15.4°, 16.0°, 17.3°, 17.9°, 19.0°, 19.7°, 20.3°, 21.0°, 21.4°, 22.5°, 24.2°, 25.8°, 26.5°, 27.0°, 29.7°, 30.2° and 30.9°±0.2 2θ and having X-ray powder diffraction pattern substantially the same as that shown in FIG. 1.

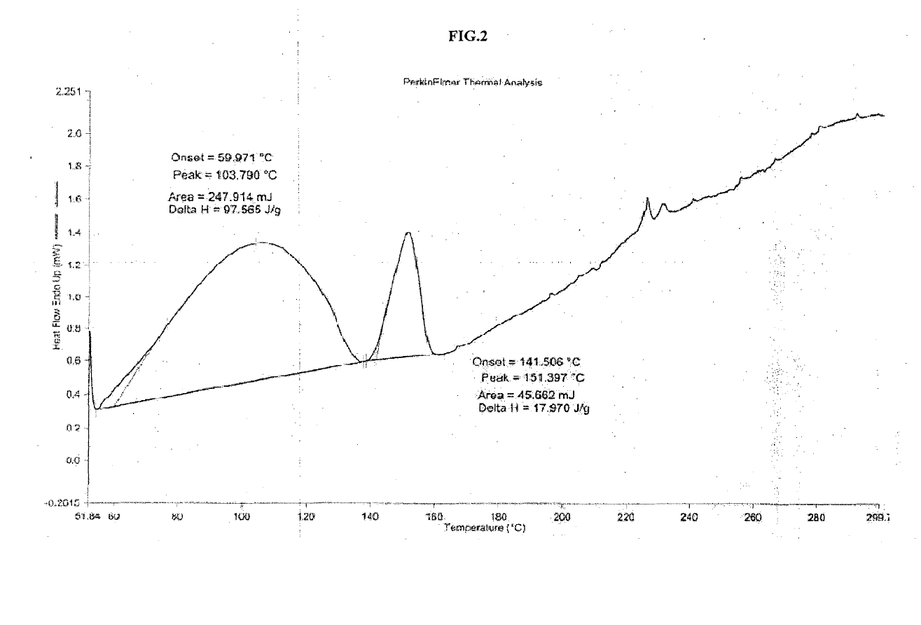

In general, the crystalline form of apixaban of Formula (I) is characterized by a differential scanning calorimetry having endothermic peak at about 103±5° C. and at about 151±5° C. and differential scanning calorimetry substantially the same as that shown in FIG. 2.

In another general aspect, there is provided an intermediate of apixaban comprising compounds of Formula (IIA), (IIB) and (IVA). In another general aspect, there is provided an isolated compound of Formula (IIA), (IIB) and (IVA).

In another general aspect, the isolated intermediates are compounds of Formula (II), Formula (IIA), Formula (IIB) and Formula (IVA).

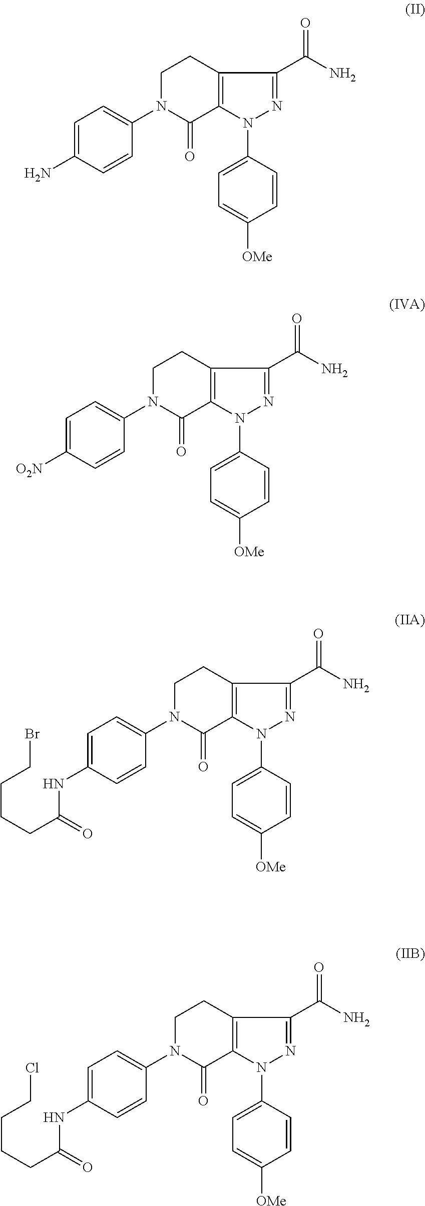 [SMILES: COc1ccc(-n2nc(C(N)=O)c3c2C(=O)N(c2ccc(N)cc2)CC3)cc1 ; COc1ccc(-n2nc(C(N)=O)c3c2C(=O)N(c2ccc([N+](=O)[O-])cc2)CC3)cc1 ; COc1ccc(-n2nc(C(N)=O)c3c2C(=O)N(c2ccc(NC(=O)CCCCBr)cc2)CC3)cc1 ; COc1ccc(-n2nc(C(N)=O)c3c2C(=O)N(c2ccc(NC(=O)CCCCCl)cc2)CC3)cc1]

In another general aspect, there is provided a crystalline form of compound (IV) characterized by X-ray powder diffraction pattern having characteristic peaks expressed in degrees 2θ (±0.2° 20) at 3.8°, 7.5°, 13.5°, 18.6°, 19.8°, 21.7°, 23.8°, and 25.5° 2θ.

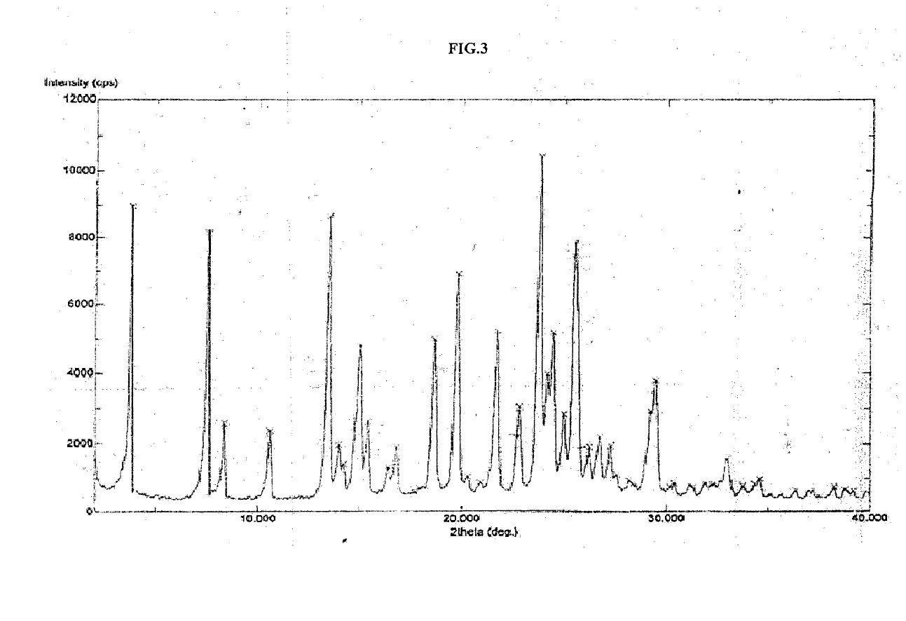

In general, crystalline form of compound (IV) is further characterized by X-ray powder diffraction pattern having characteristic peaks expressed in degrees 2θ (±0.2° 2θ) at 3.8°, 7.5°, 13.5°, 15.0°, 15.4°, 18.6°, 19.8°, 21.7°, 22.8°, 23.8°, 24.1°, 24.4°, 25.0°, 25.5°, 29.2° and 29.4° 2θ and having the X-ray powder diffraction pattern substantially the same as that shown in FIG. 3.

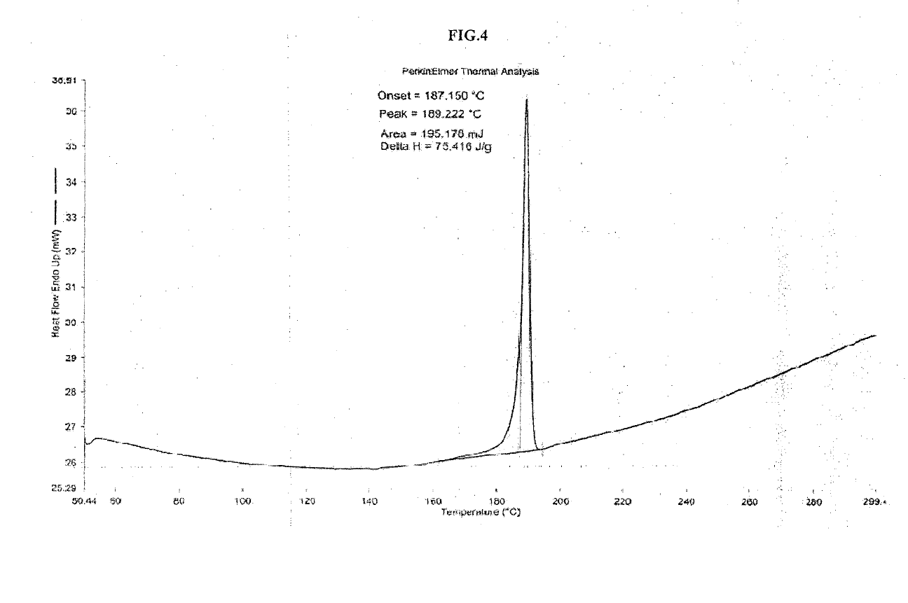

In general, crystalline form of compound (IV) is characterized by a differential scanning calorimetry having endothermic peak at about 189±5° C. and differential scanning calorimetry substantially the same as that shown in FIG. 4.

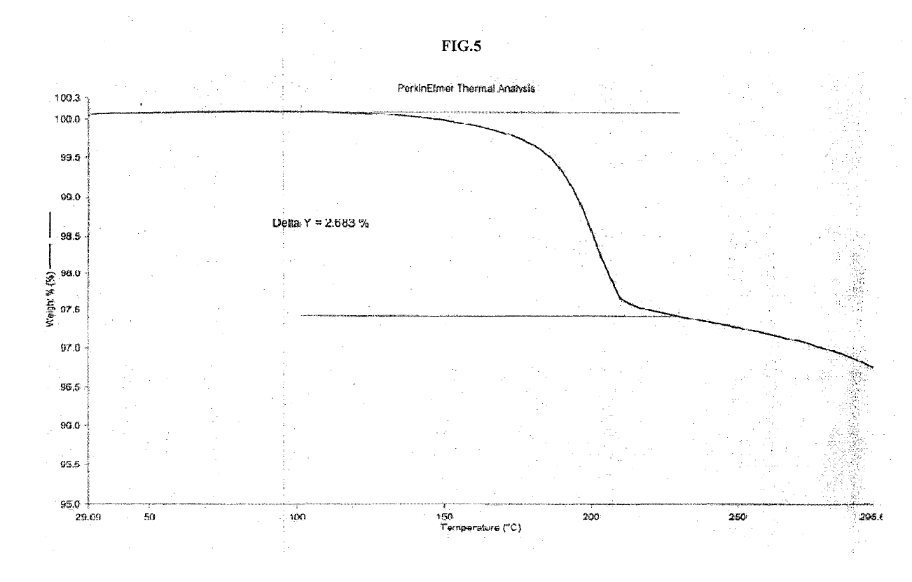

In general, crystalline form of compound (IV) is further characterized by thermogravimetric analysis substantially the same as that shown in FIG. 5.

In another general aspect, there is provided a crystalline form of compound (IVA) characterized by X-ray powder diffraction pattern having characteristic peaks expressed in degrees 2θ (±0.2° 20) at 10.8°, 15.5°, 18.6°, 20.1°, 22.6°, 24.0°, and 27.4° 2θ.

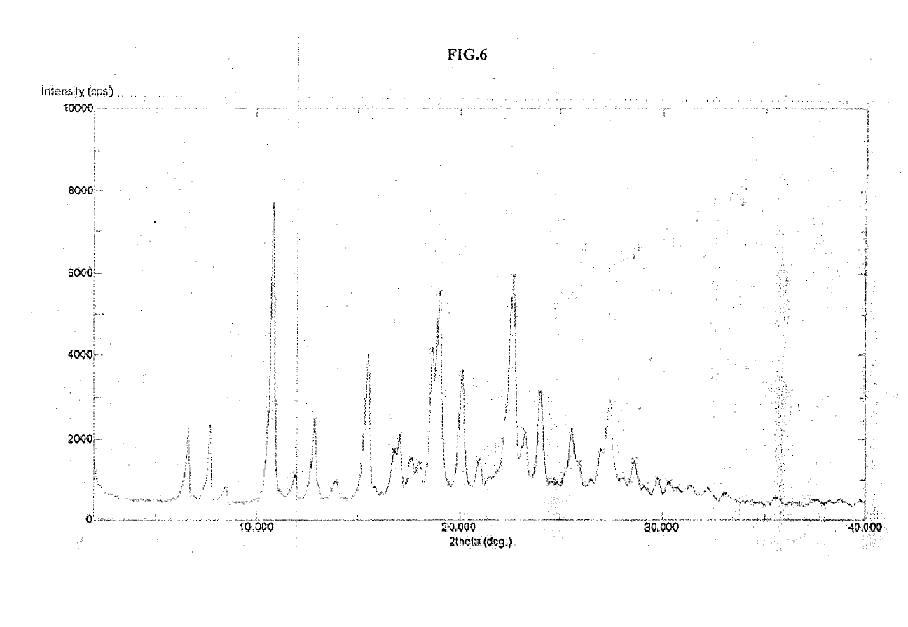

In general, the crystalline form of compound (WA) is further characterized by X-ray powder diffraction pattern having characteristic peaks expressed in degrees 2θ (±0.2° 20) at 6.6°, 7.7°, 10.8°, 12.8°, 15.5°, 17.0°, 18.6°, 19.0°, 20.1°, 22.6°, 23.2°, 24.0°, 25.5° and 27.4° 2θ and having X-ray powder diffraction pattern substantially the same as that shown in FIG. 6.

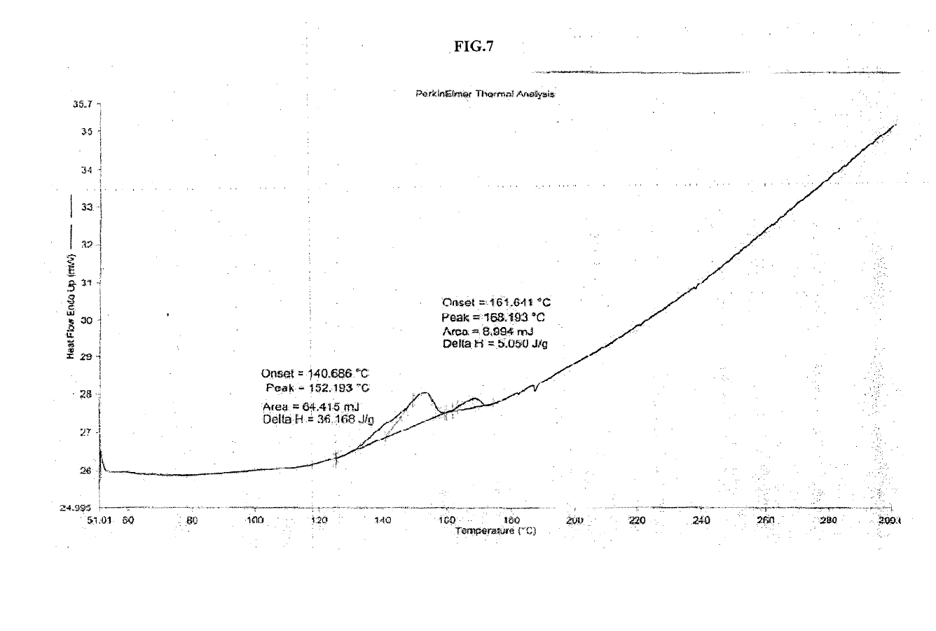

In general, the crystalline form of compound (IVA) is further characterized by a differential scanning calorimetry having endothermic peak at about 152±5° C. and at about 168±5° C. and differential scanning calorimetry substantially the same as that shown in FIG. 7.

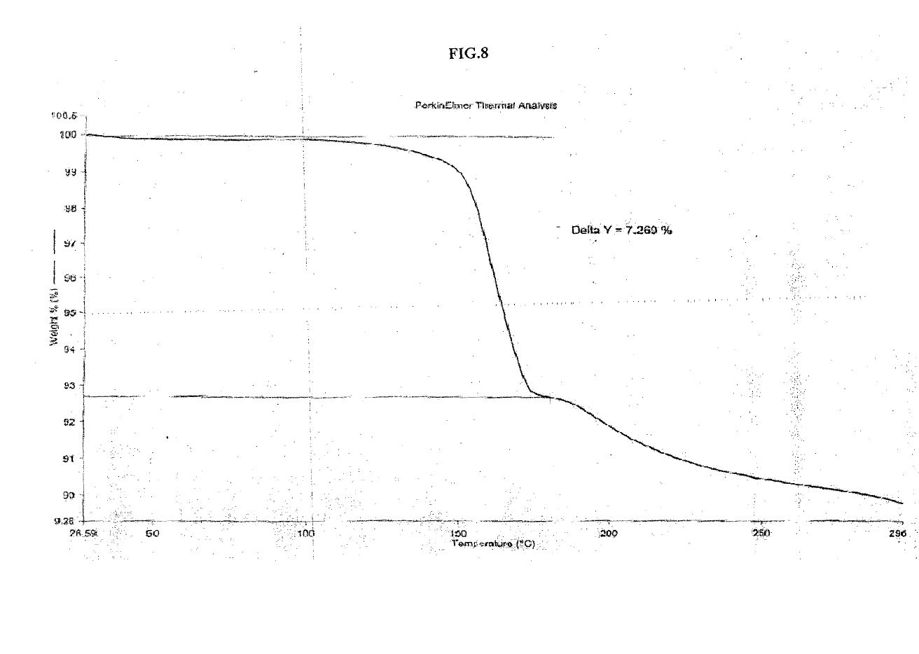

In general, the crystalline form of compound (IVA) is further characterized by a thermogravimetric analysis substantially the same as that shown in FIG. 8.

In another general aspect, there is provided a substantially amorphous form of compound (III) characterized by X-ray powder diffraction pattern having characteristic peaks expressed in degrees 2θ (±0.2° 20) at 4.8°, 9.4°, and 24.5° 2θ.

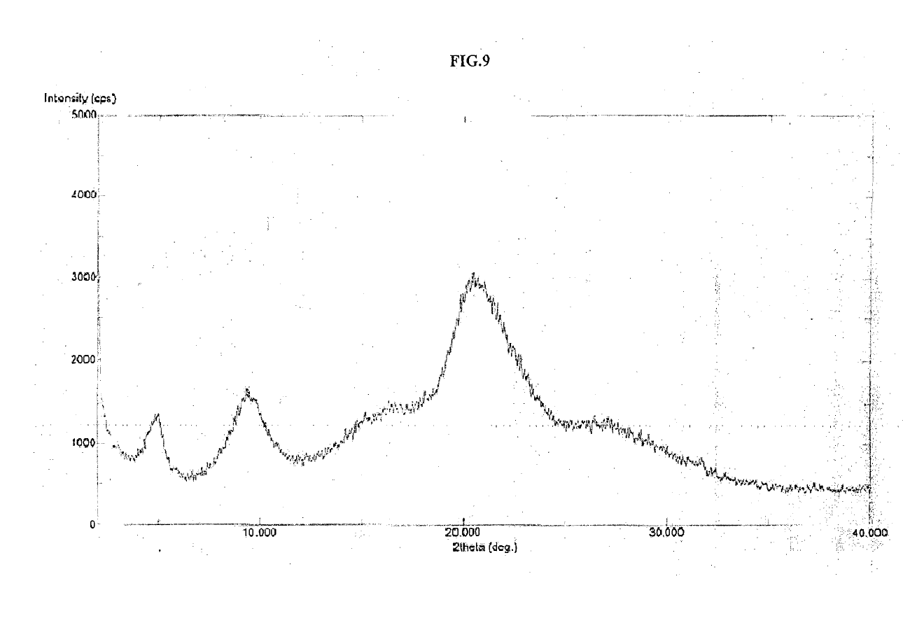

In general, the substantially amorphous form of compound (III) is further characterized by X-ray powder diffraction pattern having X-ray powder diffraction pattern substantially the same as that shown in FIG. 9.

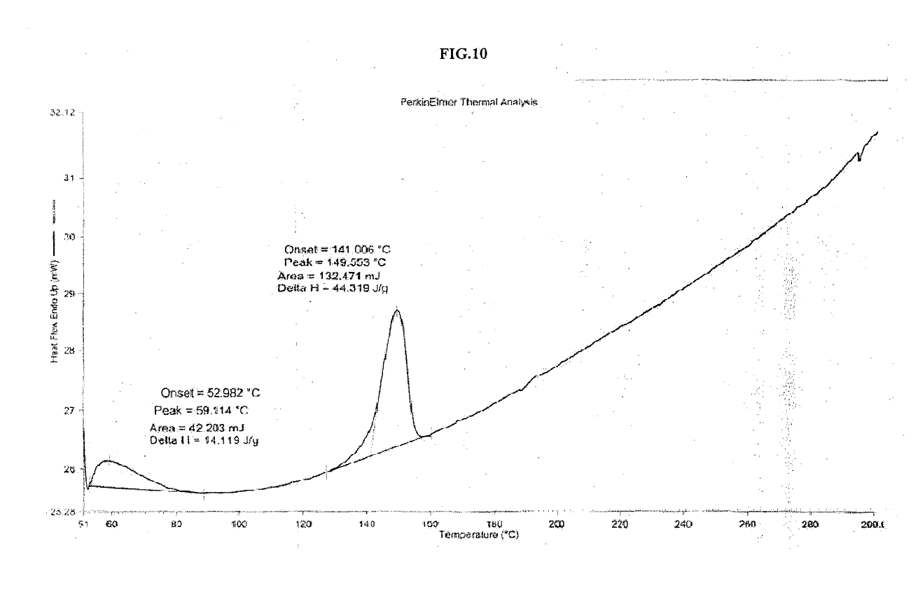

In general, the crystalline form of compound (III) is further characterized by a differential scanning calorimetry having endothermic peak at about 149±5° C. and differential scanning calorimetry substantially the same as that shown in FIG. 10.

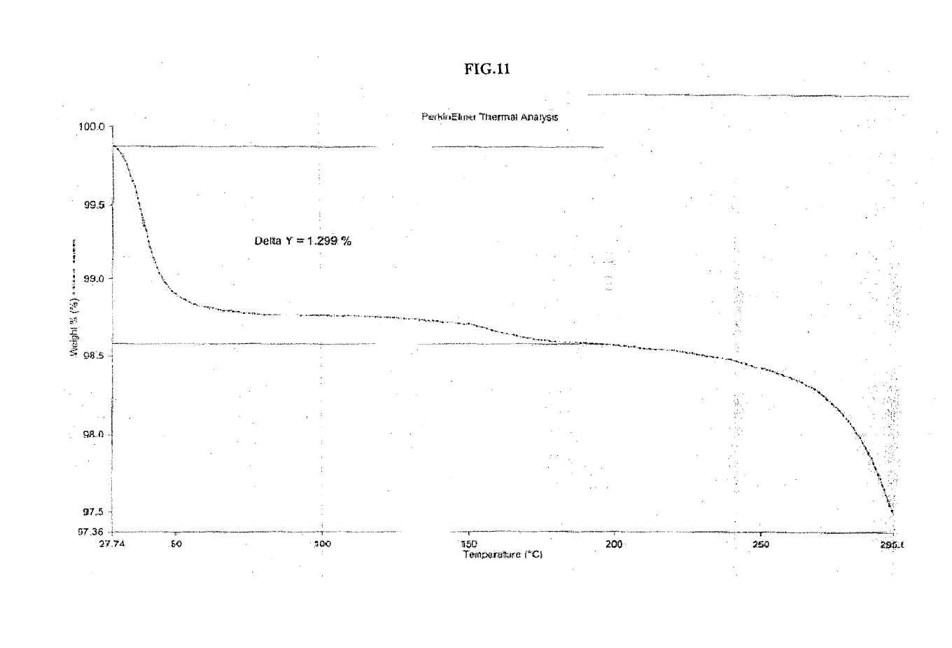

In general, the crystalline form of compound (III) is further characterized by a thermogravimetric analysis substantially the same as that shown in FIG. 11.

In another general aspect, there is provided a substantially crystalline form of compound (II) characterized by X-ray powder diffraction pattern having characteristic peaks expressed in degrees 2θ (±0.2° 20) at 18.4°, 21.2°, 22.4°, and 23.6° 2θ.

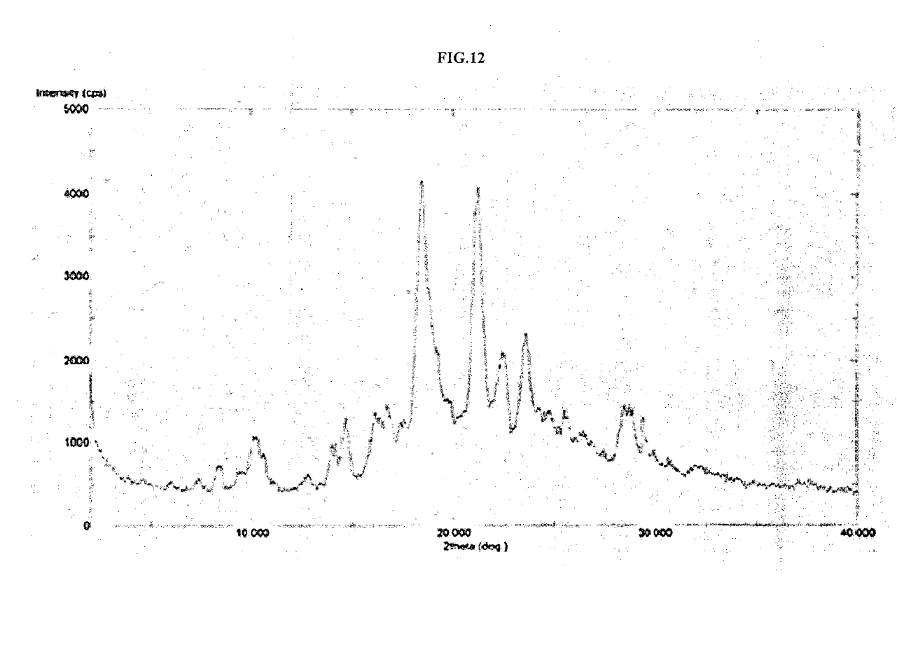

In general, the substantially crystalline form of compound (II) is further characterized by X-ray powder diffraction pattern having characteristic peaks expressed in degrees 2θ (±0.2° 2θ) at 10.1°, 10.5°, 14.0°, 14.7°, 16.2°, 16.7°, 17.5°, 18.4°, 18.9°, 19.8°, 20.3°, 21.2°, 22.4°, 23.6°, 24.8°, 25.5°, 26.3°, 28.4° and 28.8° 2θ and having X-ray powder diffraction pattern substantially the same as that shown in FIG. 12.

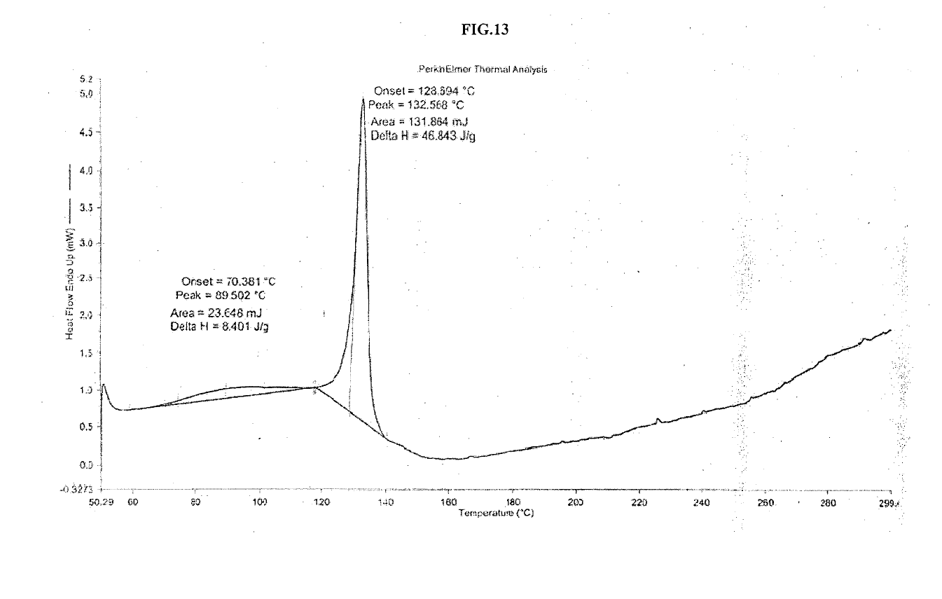

In general, the crystalline form of compound (II) is further characterized by a differential scanning calorimetry having endothermic peak at about 132±5° C. and differential scanning calorimetry substantially the same as that shown in FIG. 13.

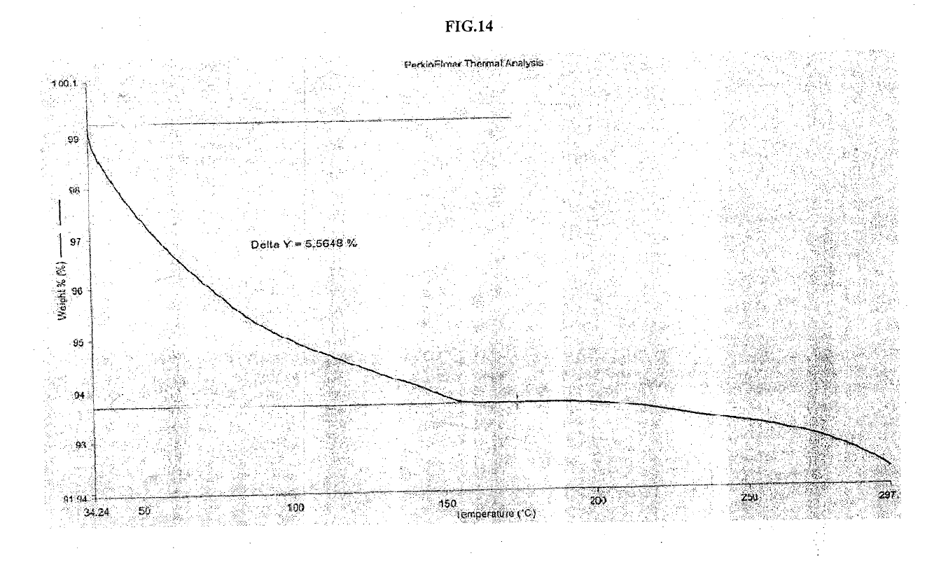

In general, the crystalline form of compound (II) is further characterized by a thermogravimetric analysis substantially the same as that shown in FIG. 14.

In another general aspect, there is provided an improved process for the preparation of apixaban of Formula (I)

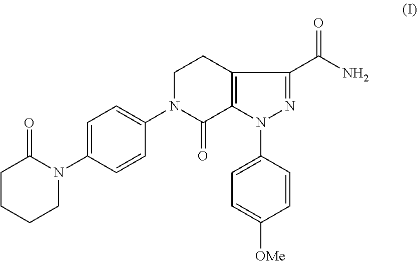 [SMILES: COc1ccc(-n2nc(C(N)=O)c3c2C(=O)N(c2ccc(N4CCCCC4=O)cc2)CC3)cc1]

the process comprising:
(a) reacting (Z)-ethyl 2-chloro-2-(2-(4-methoxyphenyl)hydrazono)acetate of Formula (V) with 3-morpholino-1-(4-nitrophenyl)-5,6-dihydropyridin-2(1H)-one of Formula (VI) in one or more organic solvents in the presence of a base to obtain compound (IV);

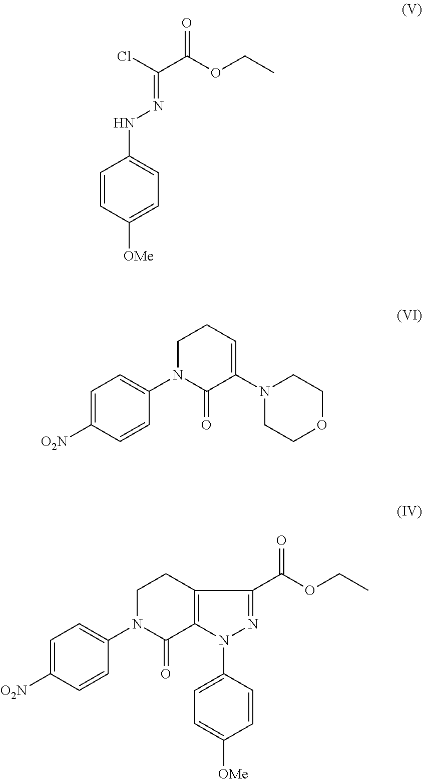 [SMILES: CCOC(=O)/C(Cl)=N/Nc1ccc(OC)cc1 ; O=C1C(N2CCOCC2)=CCCN1c1ccc([N+](=O)[O-])cc1 ; CCOC(=O)c1nn(-c2ccc(OC)cc2)c2c1CCN(c1ccc([N+](=O)[O-])cc1)C2=O]

(b) reducing the compound (IV) with a reducing agent to obtain compound (III);

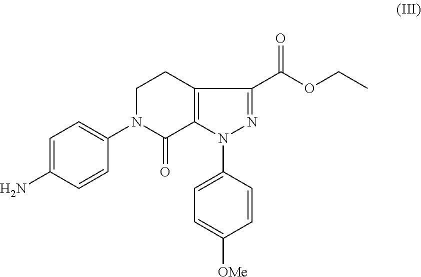 [SMILES: CCOC(=O)c1nn(-c2ccc(OC)cc2)c2c1CCN(c1ccc(N)cc1)C2=O]

(c) amidating the compound (III) with an amidating source in one or more organic solvents to obtain compound (II);

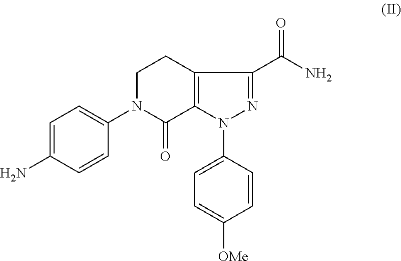 [SMILES: COc1ccc(-n2nc(C(N)=O)c3c2C(=O)N(c2ccc(N)cc2)CC3)cc1]

(d) reacting the compound (II) with 5-chlorovalaroyl chloride in the presence of a base to obtain compound (IIB);

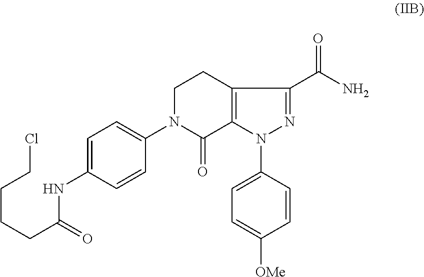 [SMILES: COc1ccc(-n2nc(C(N)=O)c3c2C(=O)N(c2ccc(NC(=O)CCCCCl)cc2)CC3)cc1]

(e) cyclizing of compound (IIB) in the presence of a base in one or more organic solvents to obtain apixaban of Formula (I); and
(f) optionally, converting apixaban of Formula (I) to an amorphous form, provided that the compound (IIB) is isolated as crystalline solid.

In general, the organic solvents comprise one or more of alcohols, nitriles, ketones, esters, ethers, amides, sulfoxide, water or mixtures thereof. In particular, alcohols comprises one or more of methanol, ethanol, n-propanol, isopropanol, and n-butanol; nitriles comprises one or more of acetonitrile, propionitrile, butyronitrile, and valeronitrile; ketones comprises one or more of acetone, methyl ethyl ketone, and methyl isobutyl ketone; esters comprises one or more of ethyl acetate, propyl acetate, isopropyl acetate, and butyl acetate; chlorinated solvents comprises one or more of methylene dichloride, chloroform, ethylene dichloride, and chlorobenzene; ethers comprises one or more of diethyl ether, diisopropyl ether, methyl tert-butyl ether, tetrahydrofuran, and dioxane; amides comprises one or more of dimethylformamide, dimethylacetamide, and N-methylformamide; sulfoxide comprises of dimethylsulfoxide.

In general, the base comprises one or more of sodium hydroxide, potassium hydroxide, lithium hydroxide, sodium carbonate, potassium carbonate, lithium carbonate, sodium bicarbonate, potassium bicarbonate, sodium hydride, potassium hydroxide, sodium methoxide, sodium ethoxide, potassium tert-butoxide, ammonia, TEA, DIPA, DEA, DIPEA, DBU, DABCO, and DBN.

The embodiments of the process involves reacting (Z)-ethyl 2-chloro-2-(2-(4-methoxyphenyl)hydrazono)acetate of Formula (V) with 3-morpholino-1-(4-nitrophenyl)-5,6-dihydropyridin-2(1H)-one of Formula (VI) in the presence of a base in one or more organic solvents.

The base comprises use of TEA or DIPEA.

In general, the reaction may be optionally performed in the presence of an alkali metal halide such as sodium iodide or potassium iodide.

In general, the organic solvents for the reaction of (Z)-ethyl 2-chloro-2-(2-(4-methoxyphenyl)hydrazono)acetate of Formula (V) with 3-morpholino-1-(4-nitrophenyl)-5,6-dihydropyridin-2(1H)-one of Formula (VI) comprises one or more of methanol, ethanol, isopropanol, n-butanol, ethyl acetate, isopropyl acetate, butyl acetate, acetone, methylethyl ketone, methylisobutyl ketone, acetonitrile, dimethylformamide, dimethylacetamide, dimethylsulfoxide, N-methyl pyrrolidone, tetrahydrofuran, 2-methyl tetrahydrofuran, toluene, xylene, methylene dichloride, and ethylene dichloride. In particular, ethyl acetate and dimethylformamide may be used to obtain the compound (IV).

In another general aspect, the reaction of (Z)-ethyl 2-chloro-2-(2-(4-methoxyphenyl)hydrazono)acetate of Formula (V) with 3-morpholino-1-(4-nitrophenyl)-5,6-dihydropyridin-2(1H)-one of Formula (VI) may be optionally performed in a biphasic solvent medium in the presence of a base and a phase transfer catalyst to obtain the compound (IV). The solvent medium comprises one or more of water-toluene, water-xylene, water-ethylacetate, methanol-cyclohexane, and water-methylene dichloride.

In general, the phase transfer catalyst comprises tetrabutyl ammonium bromide (TBAB), tetrabutyl ammonium iodide (TBAI), benzyl triethyl ammonium chloride (TEBAC), polyethylene Glycol (PEG-200, 400, 600, 800, 1000), crown ethers such as 12-crown-4, 15-crown-5, 18-crown-6, dibenzo-18-crown-6, and diaza-18-crown-6. In particular, the phase transfer catalyst may be TBAB.

In general, the reducing agent comprises one or more of Raney Nickel, Pd/C, Pt/C, Platinum oxide, Fe—HCl, Fe—NH₄Cl, Sn—HCl, and Na₂S_{X}. In particular, Fe—NH₄Cl may be used.

In general, the reduction of compound (IV) is done in one or more organic solvents comprises of methanol, ethanol, isopropanol, ethyl acetate, isopropyl acetate, acetonitrile, acetone, methylene dichloride, tetrahydrofuran, and water or mixture thereof. In particular, water, methanol, ethanol, acetone, ethyl acetate, methylene dichloride, water-methanol or water-ethanol, water-acetone, methanol-tetrahydrofuran may be used.

In general, the amidating source comprises contacting the ester compound (III) with a formamide and a base in the presence in one or more organic solvents or ammonia.

The formamide comprises N-ethyl-formamide, N-methyl-formamide, and formamide. The base comprises one or more of sodium carbonate, potassium carbonate, cesium carbonate, sodium bicarbonate, potassium bicarbonate, sodium hydride, potassium hydride, sodium hydroxide, potassium hydroxide, lithium hydroxide, sodium methoxide, sodium ethoxide, and potassium tert-butoxide. In particular, sodium methoxide may be used.

Alternatively, the amidation is also done using ammonia in presence of one or more organic solvents comprises of alcohols, ketones or esters. In particular, methanol, ethanol, isopropanol, butanol, acetone, methyl ethyl ketone, methyl isobutyl ketone, ethyl acetate, isopropyl acetate, and butyl acetate may be used.

The embodiments of the process further comprise, reacting the compound (II) with 5-chlorovalaroyl chloride in the presence of a base to obtain compound (IIB). Alternatively, the amide compound (II) may also be reacted with 5-bromovalaroyl chloride in the presence of a base to obtain compound (IIA) in one or more organic solvents.

In general, the base comprises one or more of sodium hydroxide, potassium hydroxide, lithium hydroxide, sodium carbonate, potassium carbonate, lithium carbonate, sodium bicarbonate, potassium bicarbonate, sodium hydride, potassium hydroxide, sodium methoxide, sodium ethoxide, potassium tert-butoxide, ammonia, TEA, DIPA, DEA, DIPEA, DBU, DABCO, and DBN. In particular, TEA or DIPEA may be used.

In general, the organic solvents for the reaction of 5-chlorovalaroyl chloride or 5-bromovalaroyl chloride comprises one or more of methanol, ethanol, isopropanol, n-butanol, ethyl acetate, isopropyl acetate, butyl acetate, acetone, methylethyl ketone, methylisobutyl ketone, acetonitrile, dimethylformamide, dimethyl-acetamide, dimethylsulfoxide, N-methyl pyrrolidone, tetrahydrofuran, 2-methyl tetrahydrofuran, toluene, xylene, methylene dichloride, and ethylene dichloride. In particular, tetrahydrofuran may be used.

The embodiment of the process comprises obtaining the compound of Formula (IIA) or (IIB) by addition of water to the reaction mixture. The compounds of Formula (IIA) or (IIB) may be obtained by filtration of the reaction mixture.

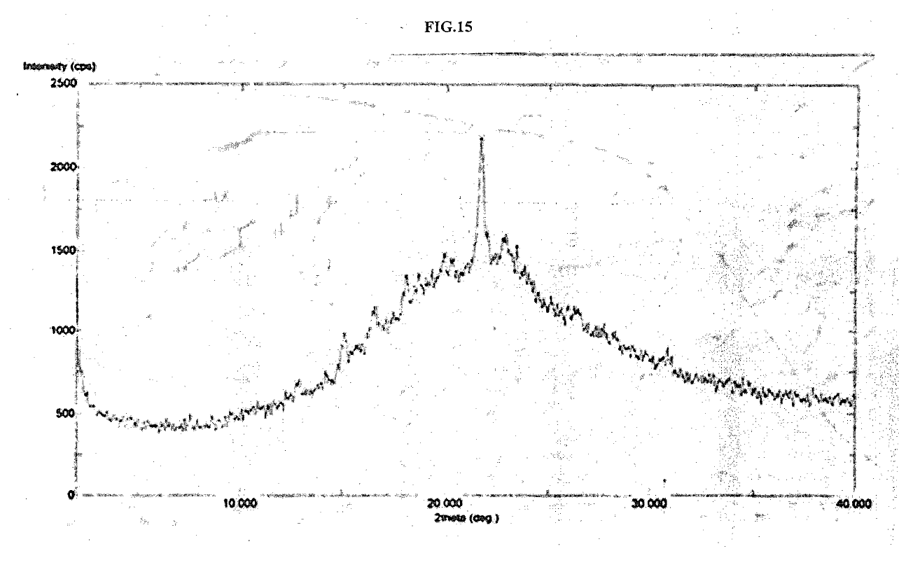

In another general aspect, there is provided a substantially amorphous form of compound (IIA) characterized by X-ray powder diffraction pattern having substantially the same as that shown in FIG. 15.

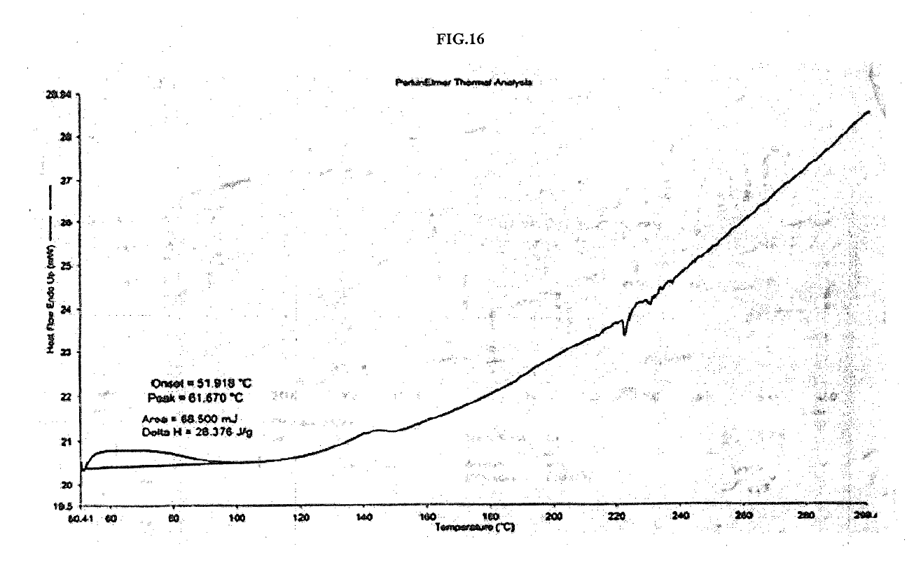

In general, the substantially amorphous form of compound (IIA) is further characterized by a differential scanning calorimetry substantially the same as that shown in FIG. 16.

In another general aspect, there is provided a crystalline form of compound (NB) characterized by X-ray powder diffraction pattern having characteristic peaks expressed in degrees 2θ (±0.2° 2θ) at 7.9°, 10.9°, 15.8°, 16.2°, 19.6°, 21.8°, and 28.9° 2θ.

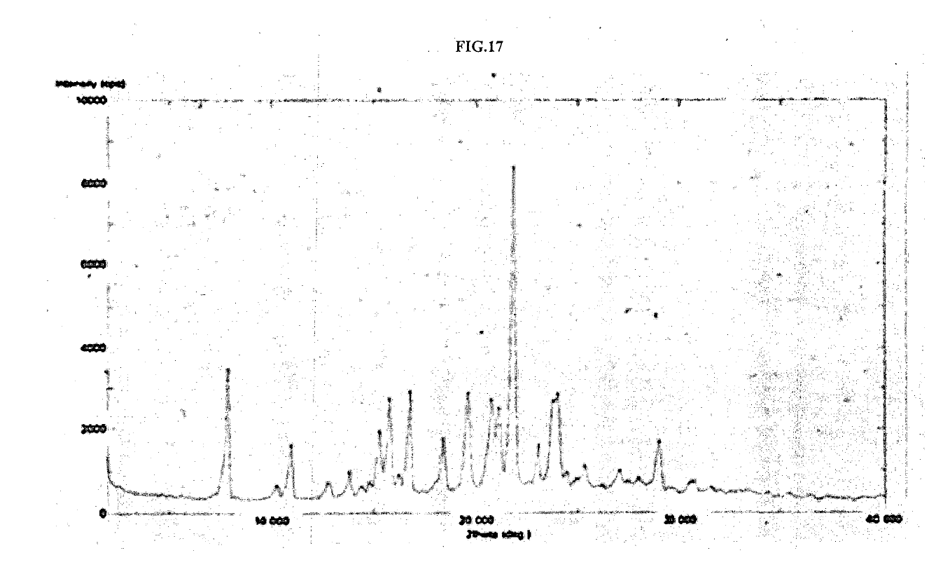

In general, the crystalline form of compound (IIB) is further characterized by X-ray powder diffraction pattern having characteristic peaks expressed in degrees 2θ (±0.2° 2θ) at 7.9°, 10.9°, 13.8°, 15.3°, 15.8°, 16.2°, 16.8°, 18.4°, 19.6°, 20.8°, 21.0°, 21.8°, 23.8°, 24.0°, and 28.9° 2θ and having X-ray powder diffraction pattern substantially the same as that shown in FIG. 17.

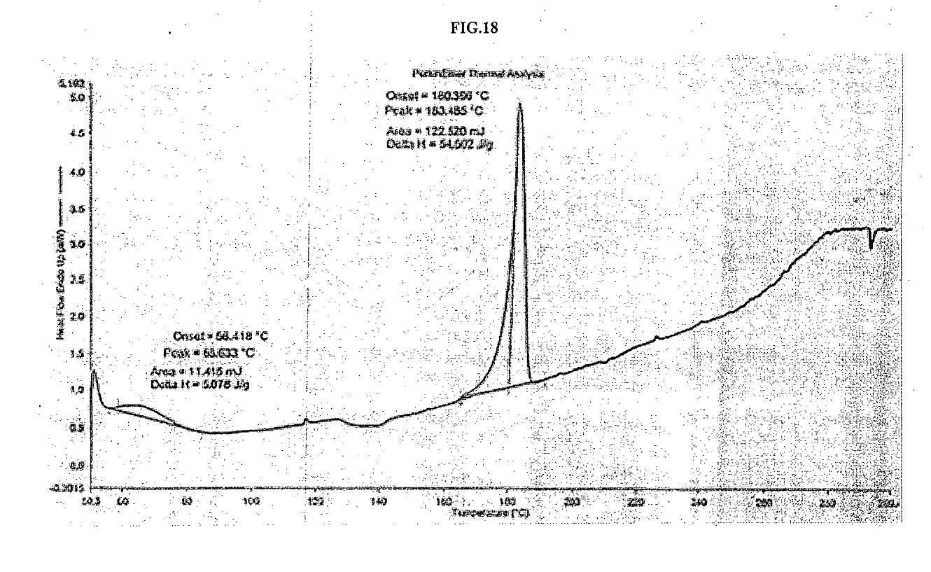

In general, the crystalline form of compound (IIB) is further characterized by a differential scanning calorimetry having endothermic peak at about 183±5° C. and differential scanning calorimetry substantially the same as that shown in FIG. 18.

In another general aspect, there is provided an improved process for the preparation of apixaban of Formula (I)

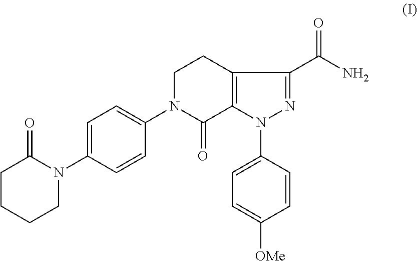 [SMILES: COc1ccc(-n2nc(C(N)=O)c3c2C(=O)N(c2ccc(N4CCCCC4=O)cc2)CC3)cc1]

the process comprising:
(a) reacting (Z)-ethyl 2-chloro-2-(2-(4-methoxyphenyl)hydrazono)acetate of Formula (V) with 3-morpholino-1-(4-nitrophenyl)-5,6-dihydropyridin-2(1H)-one of Formula (VI) in one or more organic solvents in the presence of a base to obtain compound (IV);

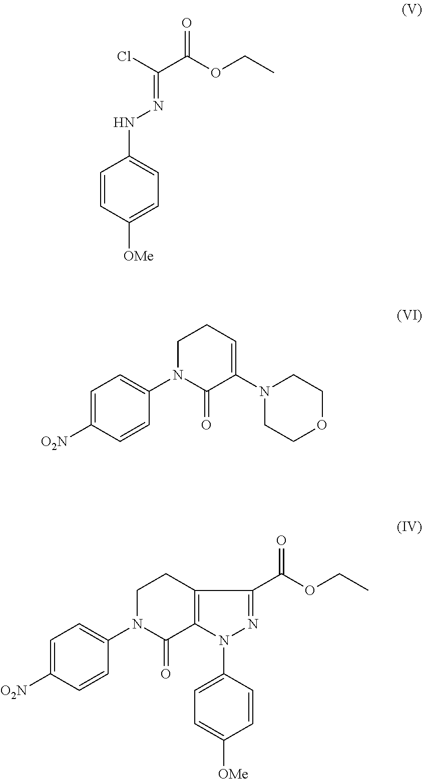 [SMILES: CCOC(=O)/C(Cl)=N/Nc1ccc(OC)cc1 ; O=C1C(N2CCOCC2)=CCCN1c1ccc([N+](=O)[O-])cc1 ; CCOC(=O)c1nn(-c2ccc(OC)cc2)c2c1CCN(c1ccc([N+](=O)[O-])cc1)C2=O]

(b) amidating the compound (IV) with an amidating source in one or more organic solvents to obtain compound (IVA);

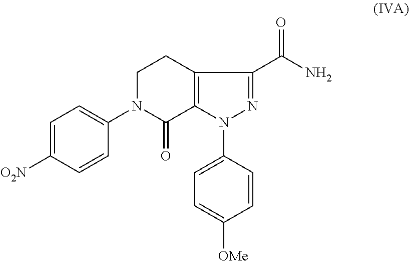 [SMILES: COc1ccc(-n2nc(C(N)=O)c3c2C(=O)N(c2ccc([N+](=O)[O-])cc2)CC3)cc1]

(c) reducing the compound (IVA) with a reducing agent to obtain compound (II);

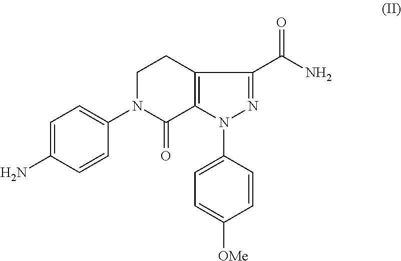 [SMILES: COc1ccc(-n2nc(C(N)=O)c3c2C(=O)N(c2ccc(N)cc2)CC3)cc1]

(d) reacting the compound (II) with 5-chlorovalaroyl chloride in the presence of a base to obtain compound (IIB);

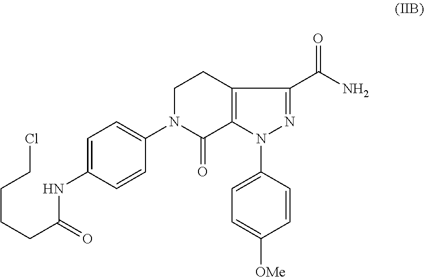 [SMILES: COc1ccc(-n2nc(C(N)=O)c3c2C(=O)N(c2ccc(NC(=O)CCCCCl)cc2)CC3)cc1]

(e) cyclizing the compound (IIB) in the presence of a base in one or more organic solvents to obtain apixaban of Formula (I); and
(f) optionally, converting apixaban of Formula (I) to an amorphous form, provided that the compound (IIB) is isolated as crystalline solid.

In another general aspect, there is provided an improved process for the preparation apixaban of Formula (I)

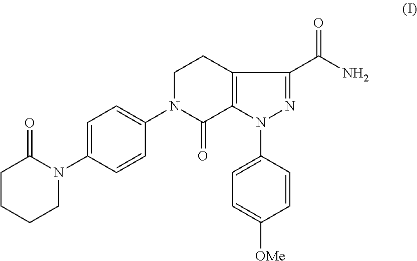 [SMILES: COc1ccc(-n2nc(C(N)=O)c3c2C(=O)N(c2ccc(N4CCCCC4=O)cc2)CC3)cc1]

the process comprising:
(a) reacting a compound (III) with an amidating source to obtain compound of Formula (II)

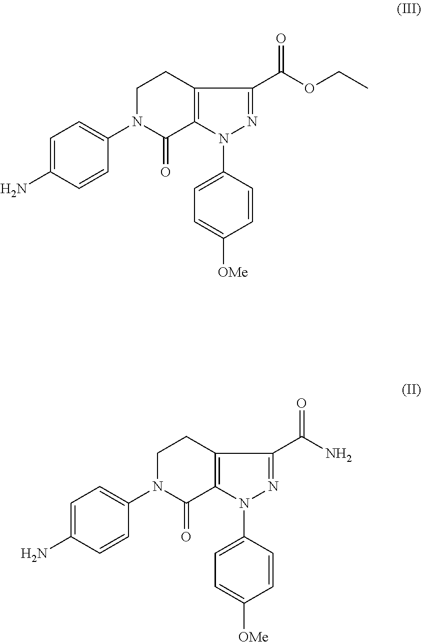 [SMILES: CCOC(=O)c1nn(-c2ccc(OC)cc2)c2c1CCN(c1ccc(N)cc1)C2=O ; COc1ccc(-n2nc(C(N)=O)c3c2C(=O)N(c2ccc(N)cc2)CC3)cc1]

(b) reacting the compound (II) with 5-chlorovalaroyl chloride in the presence of a base to obtain compound (IIB);
(c) optionally, isolating the compound (IIB) as crystalline solid; and
(d) in-situ cyclizing the compound (IIB) to obtain the apixaban of Formula (I).

In general, the amidating source comprises contacting the ester compound (III) with a formamide and a base in the presence in one or more organic solvents or ammonia.

The formamide comprises N-ethyl-formamide, N-methyl-formamide, and formamide. The base comprises one or more of sodium carbonate, potassium carbonate, cesium carbonate, sodium bicarbonate, potassium bicarbonate, sodium hydride, potassium hydride, sodium hydroxide, potassium hydroxide, lithium hydroxide, sodium methoxide, sodium ethoxide, and potassium tert-butoxide. In particular, sodium methoxide may be used.

Alternatively, the amidation is also done using ammonia in presence of one or more organic solvents comprises of alcohols, ketones or esters. In particular, methanol, ethanol, isopropanol, butanol, acetone, methyl ethyl ketone, methyl isobutyl ketone, ethyl acetate, isopropyl acetate, and butyl acetate may be used.

The embodiments of the process further comprise, reacting the compound (H) with 5-chlorovalaroyl chloride in the presence of a base to obtain compound (IIB). Alternatively, the amide compound (II) may also be reacted with 5-bromovalaroyl chloride in the presence of a base to obtain compound (IIA) in one or more organic solvents.

In, general, the base comprises one or more of sodium hydroxide, potassium hydroxide, lithium hydroxide, sodium carbonate, potassium carbonate, lithium carbonate, sodium bicarbonate, potassium bicarbonate, sodium hydride, potassium hydroxide, sodium methoxide, sodium ethoxide, potassium tert-butoxide, ammonia, TEA, DIPA, DEA, DIPEA, DBU, DABCO, and DBN. In particular, TEA or DIPEA may be used.

In general, the organic solvents for the reaction of 5-chlorovalaroyl chloride or 5-bromovalaroyl chloride comprises one or more of methanol, ethanol, isopropanol, n-butanol, ethyl acetate, isopropyl acetate, butyl acetate, acetone, methylethyl ketone, methylisobutyl ketone, acetonitrile, dimethylformamide, dimethyl-acetamide, dimethylsulfoxide, N-methyl pyrrolidone, tetrahydrofuran, 2-methyl tetrahydrofuran, toluene, xylene, methylene dichloride, and ethylene dichloride. In particular, tetrahydrofuran may be used.

The embodiment of the process comprises obtaining the compound of Formula (IIA) or (IIB) by addition of water to the reaction mixture. The compounds of Formula (IIA) or (IIB) may be obtained by filtration of the reaction mixture.

In general, there is provided reduction of compound of Formula (IV) and compound of Formula (IVA) by a reducing agent. The reducing agent comprises one or more of Raney Nickel, Pd/C, Pt/C, Platinum oxide, Fe—HCl, Fe—NH₄Cl, Sn—HCl, and Na₂S_{X}. In particular, Fe—NH₄Cl may be used.

In another general aspect, there is provided a crystalline apixaban prepared by the process of the present invention having purity of at least about 99% by area percentage of HPLC. In particular, the crystalline apixaban having purity of at least about 99.5%, or having purity of at least about 99.8%, or having purity of at least about 99.9% by area percentage of HPLC.

In another general aspect, there is provided crystalline apixaban prepared by the process of the present invention having a particle size distribution having (D₁₀) of about 50 μm or less, (D₅₀) of about 100 μm or less and (D₉₀) of about 150 μm or less. In further aspect, the apixaban may be micronized to achieve the better particle size distribution in order to make suitable Formulation.

In another general aspect, there is provided an amorphous apixaban prepared by the process of the present invention having purity of at least about 99% by area percentage of HPLC. In particular, the amorphous apixaban having purity of at least about 99.5%, or having purity of at least about 99.8%, or having purity of at least about 99.9% by area percentage of HPLC.

In general, the apixaban prepared by the process of present invention may be converted to an amorphous form by the process disclosed herein after or by the process disclosed in WO 2013/164839 A2.

In another general aspect, there is provided a composition comprising an amorphous form of apixaban. In particular, the composition is a solid dispersion that includes apixaban and a polymer.

In general, the polymer is a non-ionic polymer or an ionic polymer. The polymer comprises of hydroxypropylmethyl cellulose acetate succinate, hydroxypropylmethyl cellulose, methacrylic acid copolymers, and polyvinylpyrrolidone (PVP). In particular, PVP of different grades such as K-15, K-30, K-60, K-90 and K-120 may be used for the preparation of amorphous composition. More particular, hydroxypropylmethyl cellulose acetate succinate and PVP K-30 may be used.

In some embodiments, the apixaban of Formula (1) may be dispersed within a matrix formed by a polymer in its solid state such that it is immobilized in its amorphous form. The polymer may prevent intramolecular hydrogen bonding or weak dispersion forces between two or more drug molecules of apixaban. The solid dispersion provides for a large surface area, thus further allowing for improved dissolution and bioavailability of apixaban.

In some embodiments, the ratio of the amount of weight of apixaban within the solid dispersion to the amount by weight of the polymer therein is from about 1:1 to about 1:10. The composition of apixaban with polymer, preferably PVP K-30 or HPMC-AC may be prepared by using about 1:1 to about 1:10 polymers with respect to apixaban. The usage of higher molar amount of polymer increases the amorphous character of the drug substance.

In another general aspect there is provide a process for the preparation of composition of amorphous apixaban having at least one polymer, the process comprises mixing apixaban with a polymer in one or more organic solvents and obtaining amorphous composition of apixaban by removal of solvent.

The compound apixaban and a polymer (for example HPMC-AC or PVP K-30) may be dissolved in one or more organic solvents comprises of methanol, ethanol, isopropanol, acetone, and ethyl acetate. The amorphous solid dispersion may be obtained by removal of solvent (for example by spray drying, lyophilization, flash evaporation, and vacuum distillation) thereby leaving the amorphous solid dispersion precipitated in a matrix formed by the polymer.

The invention provides stable amorphous form of apixaban of Formula (I) having water content from about 0.5% to about 5% wt/wt and does not convert to any other solid form when stored at a temperature of up to about 40° C. and at a relative humidity of about 25% to about 75% for about three months or more.

In another general aspect, there is provided a pharmaceutical composition comprising crystalline apixaban characterized by X-ray powder diffraction pattern having characteristic peaks expressed in degrees 2θ (±0.2° 2θ) at 5.9°, 6.9°, 13.4°, 14.9°, 16.0°, 17.3°, 21.4°, 22.5°, 24.2°, and 25.8°±0.2 2θ together with one or more pharmaceutically acceptable excipients, diluents and carriers.

In another general aspect, there is provided a pharmaceutical composition comprising an amorphous form of apixaban together with one or more pharmaceutically acceptable carriers, excipients or diluents.

In another general aspect, there is provided a pharmaceutical composition comprising an amorphous apixaban having at least one polymer together one or more of pharmaceutically acceptable carriers, excipients or diluents

Powder X-ray diffraction of apixaban and intermediates thereof can be obtained under following conditions.

(i) Characterization by Powder X-ray diffraction: The X-ray powder diffraction spectrum was measured using X-Ray Diffractometer, D/Max-2200/PC Make or equivalent and having CuKα source.

(ii) Characterization by Differential Scanning calorimetry (DSC): Analytical method: Differential scanning calorimetric analysis was performed using a Perkin Elmer Diamond DSC control unit and a DSC 300° C. differential scanning calorimeter. 2-5 mg samples were placed in crimped aluminum pans and heated from 50° C. to 300° C. in a liquid nitrogen atmosphere at a heating rate of 10° C./minute. Zinc-Indium was used as the standard substance.

The invention also encompasses pharmaceutical compositions comprising apixaban of the invention. As used herein, the term “pharmaceutical compositions” includes pharmaceutical formulations such as tablets, pills, powders, liquids, suspensions, emulsions, granules, capsules, suppositories, and injection preparations.

Pharmaceutical compositions comprising an apixaban of the invention may be prepared by using diluents or excipients such as fillers, bulking agents, binders, wetting agents, disintegrating agents, surface active agents, and lubricants. Various modes of administration of the pharmaceutical compositions of the invention can be selected depending on the therapeutic purpose, for example tablets, pills, powders, liquids, suspensions, emulsions, granules, capsules, suppositories, or injection preparations.

In another general aspect, there is provided process for the preparation of apixaban of Formula (I) according to the reaction scheme-1 substantially as depicted herein after.

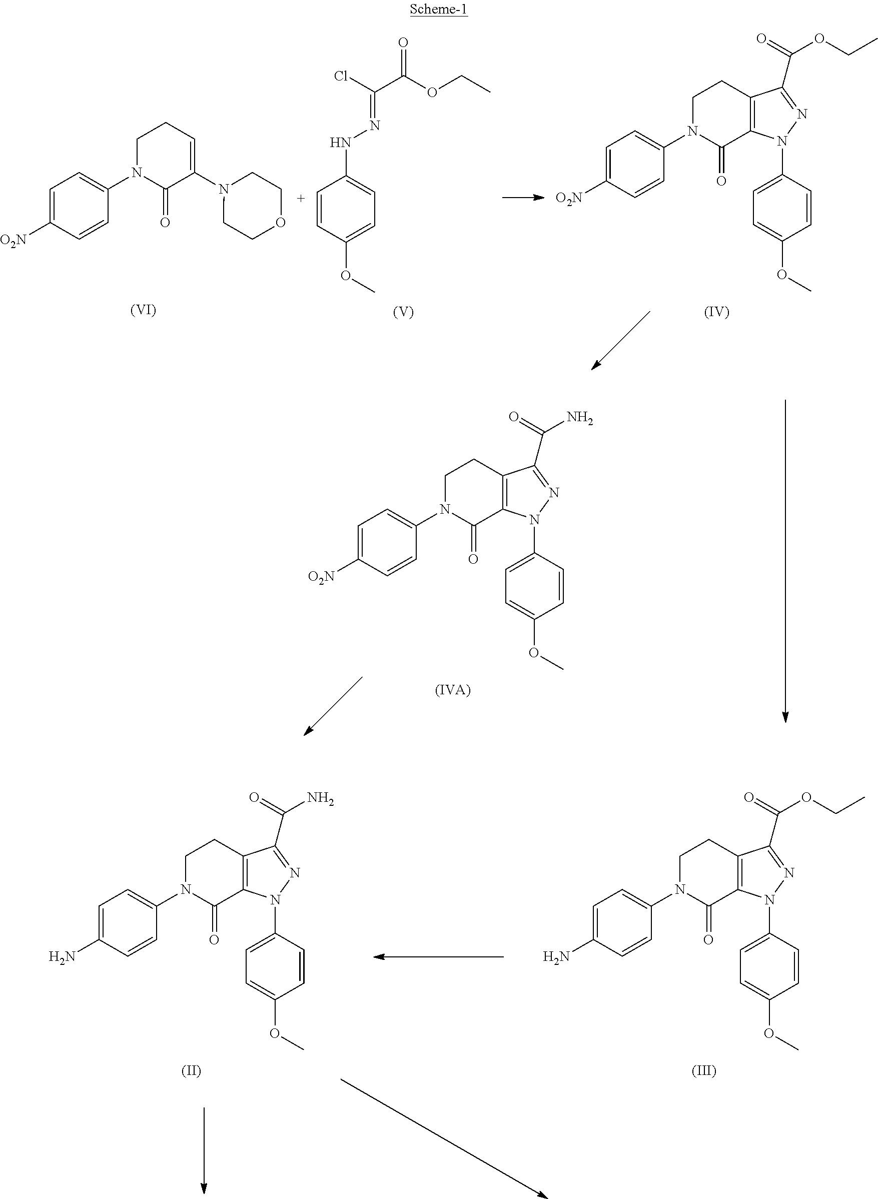

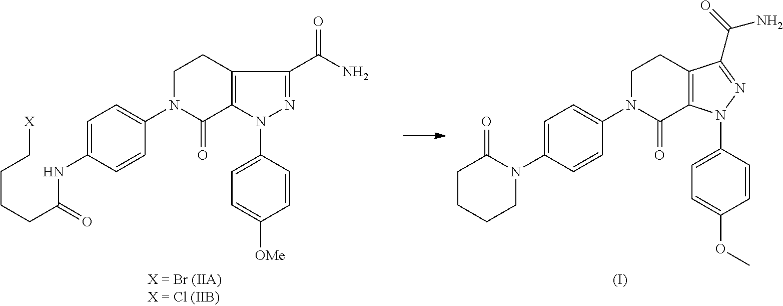

Having described the invention with reference to certain preferred embodiments, other embodiments, reaction conditions, temperature control and solvent system may become apparent to one skilled in the art from consideration of the examples provided herein after.

### Examples

### Preparation of Starting Materials

### Example-1

### Preparation of (Z)-ethyl 2-chloro-2-(2-(4-methoxyphenyl)hydrazono)acetate (V)

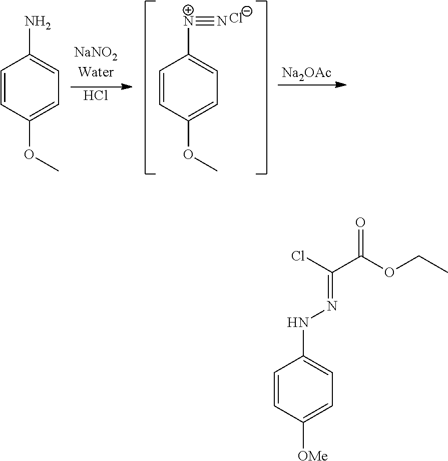

In first 500 mL 3-neck round bottom flask equipped with mechanical stirrer, thermometer and addition funnel, water (85 mL) and p-anisidine (25 gm) were added at 25° C. to 30° C. The reaction mixture was cooled to 0° C. to 5° C. Con. HCl (50 mL) was added to the reaction mixture and stirred for 15 min.

In second 500 mL 3-neck round bottom flask equipped with mechanical stirrer, thermometer and addition funnel, water (43 mL) and sodium nitrite 16.9 g were added at 25° C. to 30° C. The reaction mixture was cooled to 0° C. to 5° C. and above prepared reaction mixture was added to it. The reaction mixture was stirred for 1 hour at 0-5° C.

In third 500 mL 3-neck round bottom flask equipped with mechanical stirrer, thermometer and addition funnel, water (81 mL) and sodium acetate (38.4 g) were added at room temperature. The reaction mixture was cooled to 0° C. to 5° C. and ethyl 2-chloroaceto acetate (33.5 gm) and ethyl acetate (162.5 mL) were added and stirred for 15 min. The above reaction mixture prepared in second 500 mL 3-neck round bottom flask was added at 0° to 5° C. and stirred for 30 min. The reaction mixture was warmed to 25° C. to 30° C. for 30 mins. The separated organic layer was charcoalized and filtered. The filtrate was distilled to remove ethyl acetate under vacuum at 45° C. to 50° C. and cooled to 25° C. Methanol (50 mL) was added and cooled to 0° C. to 5° C. The reaction mixture was stirred for 30 mins and filtered. The solid obtained was washed with methanol and dried under vacuum at 45° C. to 50° C. to obtain 28.2 g of titled compound.

### Example-2

### Preparation of 3-morpholino-1-(4-nitrophenyl)-5,6-dihydropyridin-2(1H)-one (VI)

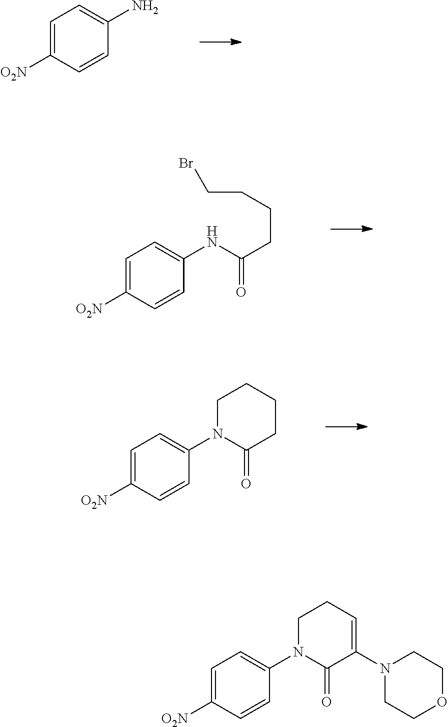 [SMILES: Nc1ccc([N+](=O)[O-])cc1 ; O=C(CCCCBr)Nc1ccc([N+](=O)[O-])cc1 ; O=C1CCCCN1c1ccc([N+](=O)[O-])cc1 ; O=C1C(N2CCOCC2)=CCCN1c1ccc([N+](=O)[O-])cc1]

### (A) Preparation of 5-bromo-N-(4-nitrophenyl)pentanamide

In 1 L 3-neck round bottom flask equipped with mechanical stirrer, thermometer and addition funnel, 4-nitroaniline (50 gm) and tetrahydrofuran (250 mL) were taken at 25° C. and cooled to 0° to 5° C. 5-bromovalaroyl chloride (101.09 gm) and a solution of triethylamine (65.81 gm) in tetrahydrofuran (50 mL) were added at 0° C. to 5° C. The reaction mixture was stirred at 25° C. for 1-2 hours and cooled to 0° C. to 5° C. The reaction mixture was stirred at 5° C. to 10° C. for 1 hour and water (1500 mL) was added. The reaction mixture was filtered and the wet-cake was washed with water and dried at 55° C. to 60° C. under vacuum for 8 hours to obtain 108.2 g of 5-bromo-N-(4-nitrophenyl)pentamide.

The reaction may be repeated to obtain 5-chloro-N-(4-nitrophenyl)pentamide by replacing 5-bromovalaroyl chloride with 5-chlorovalaroyl chloride.

### (B) Preparation of 1-(4-nitrophenyl)piperidin-2-one

In 3 L 4-neck round bottom flask equipped with mechanical stirrer, thermometer and addition funnel, 60% sodium hydride (23.9 gm) and TI-IF (450 mL) were added at 25° C. and stirred for 15 min to obtain reaction mixture. The reaction mixture was cooled to 0° C. to 5° C. and 5-bromo-N-(4-nitrophenyl)pentamide (100 gm) was added. The reaction mixture was stirred for 30 mins. Water (1250 mL) and MDC (750 mL) were added at 5° C. to 10° C. and warmed to 25° C. and stirred for 1 hour. The reaction mixture was filtered and the filtrate was distilled under vacuum at 45° C. to 50° C. Toluene (100 mL) was added and stirred for 30 min. The reaction mixture was filtered and washed with toluene. The product was dried under vacuum at 50° C. to 55° C. for 6-8 hours to obtain 52.5 gm of 1-(4-nitrophenyl)piperidin-2-one.

### (C) Preparation of 3-morpholino-1-(4-nitrophenyl)-5,6-dihydropyridin-2(1H)-one

In 3 L 4-neck round bottom flask equipped with mechanical stirrer, thermometer and addition funnel, methylene dichloride (120 mL) and PCI₅ (85.15 gm) were added at 25° C. The reaction mixture was stirred for 15 min and cooled to 0° C. to 5° C. A solution of 1-(4-nitrophenyl)piperidin-2-one (30 gm) in methylene dichloride (120 ml) was added and stirred for 30 min. The reaction mixture was raised to 25° C. to 30° C., stirred for 2 hours and cooled to 0° C. to 5° C. Water (600 mL) was added and stirred to separate the layer. The separated methylene dichloride was dried over anhydrous sodium sulphate and distilled under vacuum at 45° C. to 50° C. Morpholine (156 mL) was added and stirred for 30 min followed by heating at 125-130° C. for 30 min. The reaction mixture was cooled to 70° C. to 75° C. and distilled to remove excess morpholine under vacuum at 70° C. to 75° C. and cooled to 55° C. to 60° C. Methanol (120 mL) and water (60 mL) was added at 55° C. to 60° C. and cooled to 25° C. to 30° C. The reaction mixture was stirred for 30 mins and filtered. The solid was washed with water and dried at 50° C. to 55° C. under vacuum to obtain 16 gm of 3-morpholino-1-(4-nitrophenyl)-5,6-dihydropyridin-2(1H)-one (VI).

### Preparation of Apixaban

### Example-3

### Preparation of ethyl 1-(4-methoxyphenyl)-6-(4-nitrophenyl)-7-oxo-4,5,6,7-tetrahydro-1H-pyrazolo[3,4-c]pyridine-3-carboxylate (IV)

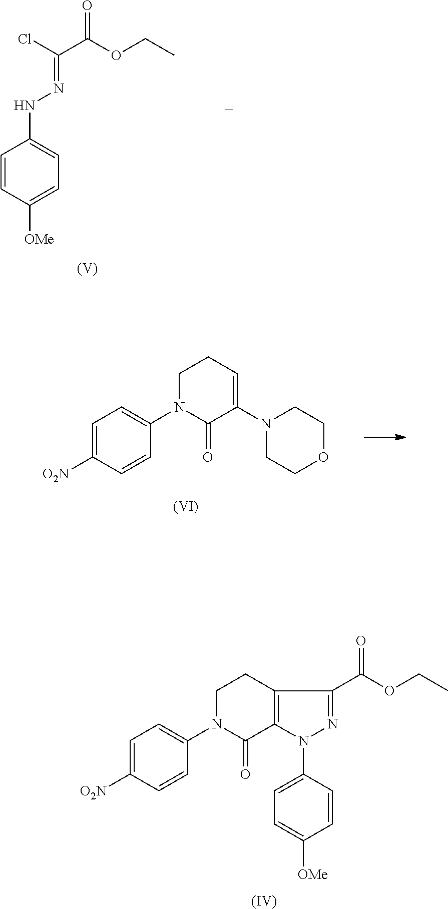 [SMILES: CCOC(=O)/C(Cl)=N/Nc1ccc(OC)cc1 ; "(V)" ; O=C1C(N2CCOCC2)=CCCN1c1ccc([N+](=O)[O-])cc1 ; "(VI)" ; CCOC(=O)c1nn(-c2ccc(OC)cc2)c2c1CCN(c1ccc([N+](=O)[O-])cc1)C2=O ; "(IV)"]

In 500 mL 3-neck round bottom flask equipped with mechanical stirrer, thermometer and addition funnel, 3-morpholino-1-(4-nitrophenyl)-5,6-dihydropyridin-2(1H)-one (VI) (8.44 g) and (Z)-ethyl 2-chloro-2-(2-(4-methoxyphenyl)hydrazono)acetate (V) (10 g) and ethyl acetate (50 mL) were added at 25° C. to 30° C. Triethylamine (6.6 g) was added to the reaction mixture and heated to 75° C. to 80° C. for 6 hours. The reaction mixture was distilled to remove ethyl acetate. The residue was diluted with water (30 mL) at 25° C. to 30° C. and 4N HCl (70 mL) solution was added. The reaction mixture was stirred for 2 hours at 25° C. and filtered. The wet-cake was washed with water and dried at 60° C. to 65° C. for 5-6 hours under vacuum to obtain 12.3 gm (86% yield) of titled compound.

### Example-4

### Preparation of 1-(4-methoxyphenyl)-6-(4-nitrophenyl)-7-oxo-4,5,6,7-tetrahydro-1H-pyrazolo[3,4-c]pyridine-3-carboxamide (IVA)

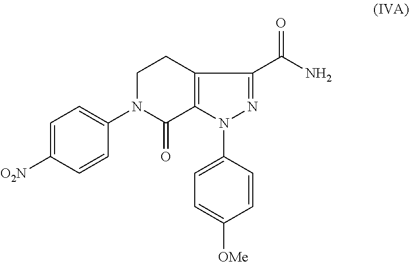 [SMILES: COc1ccc(-n2nc(C(N)=O)c3c2C(=O)N(c2ccc([N+](=O)[O-])cc2)CC3)cc1]

In 250 mL 3-neck round bottom flask equipped with mechanical stirrer, thermometer and addition funnel, compound (V) and formamide (4.43 gm) in 20 ml DMF in 250 ml 3N RBF at 25° C. Sodium methoxide (2.65 gm) and methanol (4 mL) were added to the reaction mixture and heated to 65° C. to 70° C. for 1 hour. The reaction mixture was cooled to 25° C. and water (80 mL) was added at 0-5° C. The reaction mixture was stirred at 5° C. to 10° C. for 1 hour and filtered. The reaction mixture was filtered and the wet-cake was washed with water and dried under vacuum at 60° C. to 65° C. for 6-8 hours to obtain 2.5 gm of titled compound.

### Example-5

### Preparation of ethyl 6-(4-aminophenyl)-1-(4-methoxyphenyl)-7-oxo-4,5,6,7-tetrahydro-1H-pyrazolo[3,4-c]pyridine-3-carboxylate (III)

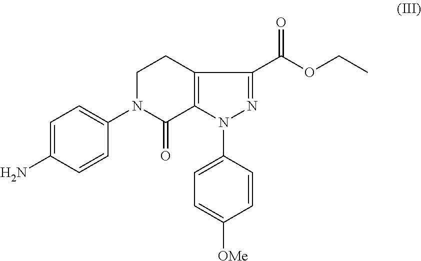 [SMILES: CCOC(=O)c1nn(-c2ccc(OC)cc2)c2c1CCN(c1ccc(N)cc1)C2=O]

In 500 mL 3-neck round bottom flask equipped with mechanical stirrer, thermometer and addition funnel, compound (IV) (23 g), iron (11.77 g) in methanol:water (7:3) (60 mL) were added at 25-30° C. Ammonium chloride (6.20 g) was added and the reaction mixture was heated at 65° C. to 70° C. for 5 hours, cooled to 25° C. to 30° C. and methylene dichloride (100 mL) was added. The reaction mixture was filtered and washed with methylene dichloride. The reaction mixture was distilled and methylene dichloride (45 mL) was added. The combined organic layer was washed with water and distilled under vacuum at 45° C. to 50° C. to obtain residue. Cyclohexane (160 mL) was added to the residue and stirred for 30 mins. The reaction mixture was filtered and wet-cake was washed with cyclohexane, dried at 50° C. to 55° C. for 3 to 4 hours under vacuum to obtain 18.4 g (86% yield) of titled compound.

### Example-6

### Preparation of 6-(4-aminophenyl)-1-(4-methoxyphenyl)-7-oxo-4,5,6,7-tetrahydro-1H-pyrazolo[3,4-c]pyridine-3-carboxamide (II)

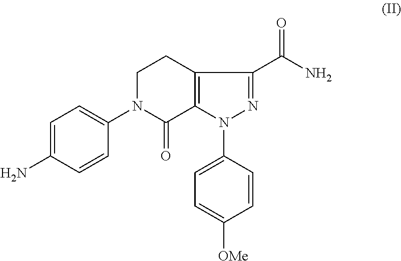 [SMILES: COc1ccc(-n2nc(C(N)=O)c3c2C(=O)N(c2ccc(N)cc2)CC3)cc1]

In 250 mL 3-neck round bottom flask equipped with mechanical stirrer, thermometer and addition funnel, compound (III) (4.0 gm) and formamide (4.43 gm) in dimethylformamide (20 mL) were added at 25° C. Sodium methoxide (2.65 gm) and methanol (4 mL) were added to the reaction mixture and heated to 65° C. to 70° C. for 1 hour. The reaction mixture was cooled to 25° C. and water (80 mL) was added at 0-5° C. The reaction mixture was stirred at 5° C. to 10° C. for 1 hour and filtered. The reaction mixture was filtered and the wet-cake was washed with water and dried under vacuum at 60° C. to 65° C. for 6-8 hours to obtain 2.5 gm of titled compound.

### Example-7

### Preparation of 6-(4-aminophenyl)-1-(4-methoxyphenyl)-7-oxo-4,5,6,7-tetrahydro-1H-pyrazolo[3,4-c]pyridine-3-carboxamide (II)

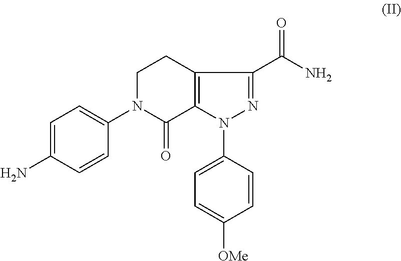 [SMILES: COc1ccc(-n2nc(C(N)=O)c3c2C(=O)N(c2ccc(N)cc2)CC3)cc1]

In 250 mL 3-neck round bottom flask equipped with mechanical stirrer, thermometer and addition funnel, compound (III) (20 g) and 18% methanolic ammonia solution (300 mL) were heated at 4 Kg pressure in autoclave at 60° C. to 65° C. for 15 hours. The reaction mixture was distilled under vacuum to obtain residue. Water (140 mL) was added and stirred for 30 min at 25° C. The reaction mixture was cooled at 5° C. to 10° C. and filtered. The wet-cake was washed with water to obtain titled compound 16.8 g (90% yield) of titled compound.

### Example-8

### Preparation of Compound (IIA)

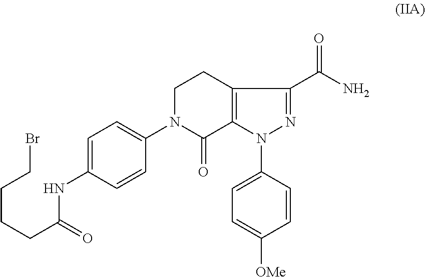 [SMILES: COc1ccc(-n2nc(C(N)=O)c3c2C(=O)N(c2ccc(NC(=O)CCCCBr)cc2)CC3)cc1]

In 250 mL 3-neck round bottom flask equipped with mechanical stirrer, thermometer and addition funnel, compound (III) (2.5 gm) and THF (25 mL) were added at 25-30° C. and cooled to 0° C. to 5° C. 5-bromovalaroyl chloride (1.85 g) and a solution of triethylamine (1.23 g) in THF (2.5 mL) was added at 0° C. to 5° C. The reaction mixture was stirred for 1 hour and water (50 mL) was added. The reaction mixture was filtered and the wet-cake was washed with water and dried at 55° C. to 60° C. under vacuum for 8 hours to obtain 5.1 g of compound (IIA).

### Example-9

### Preparation of Compound (IIB)

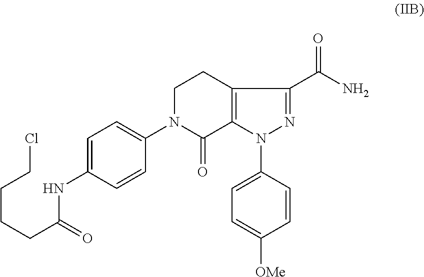 [SMILES: COc1ccc(-n2nc(C(N)=O)c3c2C(=O)N(c2ccc(NC(=O)CCCCCl)cc2)CC3)cc1]

In 250 mL 3-neck round bottom flask equipped with mechanical stirrer, thermometer and addition funnel, compound (III) (10 g), 5-chlorovalaroyl chloride (10.27 g), triethylamine (9.4 g) and THF (70 mL) were heated at 60° C. to 65° C. for 4 hours. The reaction mixture was cooled at 25° C. to 35° C. and water (200 mL) was added. The reaction mixture was distilled to remove THF and cooled to 25° C. to 35° C. The reaction mixture was stirred for 1 hour and filtered. The wet-cake was washed with water and dried to obtain 5.1 g of compound (IIB). The compound (IIB) was characterized by X-ray powder diffraction pattern (FIG. 17). The solid compound was recrystallized in ethyl acetate at 65° C. to obtain pure compound (IIB).

### Example-10

### Preparation of Apixaban (I)

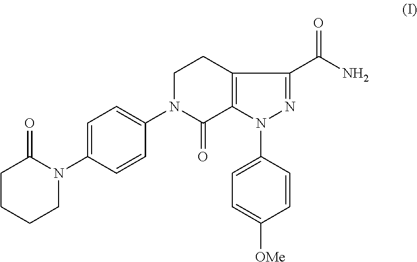 [SMILES: COc1ccc(-n2nc(C(N)=O)c3c2C(=O)N(c2ccc(N4CCCCC4=O)cc2)CC3)cc1]

In 250 mL 3-neck round bottom flask equipped with mechanical stirrer, thermometer and addition funnel, compound (III) (2.5 gm) and THF (25 mL) were added at 25-30° C. and cooled to 0° C. to 5° C. 5-bromovalaroyl chloride (1.85 g) and a solution of triethylamine (1.23 g) in THF (2.5 mL) was added at 0° C. to 5° C. The reaction mixture was stirred for 1 hour and 60% sodium hydride (1.58 gm) was added at 0° to 5° C. The reaction mixture was heated to 25° C. to 30° C. and stirred for 2 hours. Water (50 mL) was added and stirred for 1 hour. The reaction mixture was filtered and washed with water. The wet-cake was dried at 60° C. to 65° C. for 8 hours under vacuum to obtain 1.8 of apixaban.

### Example-11

### Preparation of Apixaban (I)

In 250 mL 3-neck round bottom flask equipped with mechanical stirrer, thermometer and addition funnel, compound (IIA) (5 g), THF (50 mL) and 60% sodium hydride (1.2 g) were added at 25-30° C. and cooled to 0° C. to 5° C. The reaction mixture was stirred for 2 hours. Water (20 mL) was added and stirred for 1 hour. The reaction mixture was extracted with methylene dichloride (20 mL) and organic layer was separated. The separated organic layer was distilled to obtain the residue. Cyclohexane (20 mL) was added and stirred for 30 min. The reaction mixture was filtered and the wet-cake was dried at 60° C. to 65° C. for 8 hours under vacuum to obtain 32 g of crystalline apixaban characterized by X-ray powder diffraction substantially as same as shown in FIG. 1.

### Example-12

### Preparation of Apixaban

In 250 mL 3-neck round bottom flask equipped with mechanical stirrer, thermometer and addition funnel, compound (IIA) (5 g), dimethylacetamide (50 mL) and 60% sodium hydride (1.2 g)′ were added at 25-30° C. and cooled to 0° C. to 5° C. The reaction mixture was stirred for 2 hours. 10% aqueous acetic acid was added to the reaction mixture to adjust the pH 6 to 6.5. The reaction mixture was diluted with water (100 mL) was stirred for 1 hour. The reaction mixture was filtered at 0° C. to 5° C. The wet-cake was washed with water to obtain 3.1 g of apixaban. The apixaban obtained was recrystallized in methanol at 65° C. to obtain pure apixaban.

### Example-13

### Purification of Apixaban (I)

In 250 mL 3-neck round bottom flask equipped with mechanical stirrer, thermometer and addition funnel, apixaban (I) (5 g), methylene dichloride (50 mL) and methanol (20 mL) were added at 25-30° C. The reaction mixture was heated at 40° C. to 45° C. to obtain the clear solution. Methyl tert-butyl ether (60 mL) was added to the reaction mixture. The solid obtained was filtered and washed with methyl tert-butyl ether to obtain 3.1 g apixaban.

### Preparation of Amorphous Apixaban

### Example-14

10 mg of apixaban and 15 mL methanol were taken in round bottom flask at 25-30° C. The reaction mixture was heated at 45-50° C. to obtain clear solution. 100 mg of PVP-K30 polymer was added and stirred at 45-50° C. for 2 hours. The reaction mixture was distilled under vacuum at 60-65° C. The product was dried under vacuum at 55-60° C. to obtain 120 mg amorphous apixaban.

### Example-15

100 mg of apixaban and 15 mL methanol were taken in round bottom flask at 25-30° C. The reaction mixture was heated at 45-50° C. to obtain clear solution. 200 mg of PVP-K30 polymer was added and stirred at 45-50° C. for 2 hours. The reaction mixture was distilled under vacuum at 60-65° C. The product was dried under vacuum at 55-60° C. to obtain 155 mg amorphous apixaban.

### Example-16

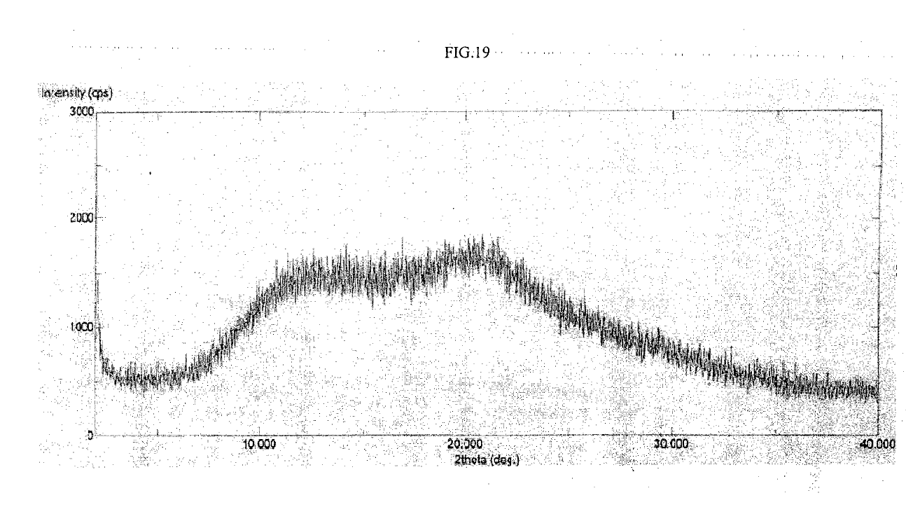

50 mg (0.108 mmol) of apixaban and 10 mL methanol were taken in round bottom flask at 25-30° C. The reaction mixture was heated at 45-50° C. to obtain clear solution. 200 mg of PVP-K30 polymer was added and stirred at 45-50° C. for 2 hours. The reaction mixture was distilled under vacuum at 60-65° C. The product was dried under vacuum at 55-60° C. to obtain 125 mg amorphous apixaban. (XRD: FIG. 19)

### Example-17

40 mg of apixaban and 10 mL methanol were taken in round bottom flask at 25-30° C. The reaction mixture was heated at 45-50° C. to obtain clear solution. 320 mg of PVP-K30 polymer was added and stirred at 45-50° C. for 2 hours. The reaction mixture was distilled under vacuum at 60-65° C. The product was dried under vacuum at 55-60° C. to obtain 145 mg amorphous apixaban. (XRD: FIG. 19).

While the present invention has been described in terms of its specific embodiments, certain modifications and equivalents will be apparent to those skilled in the art and are intended to be included within the scope of the present invention.

1.-26. (canceled)

27. A process for preparing apixaban, the process comprising:
(a) reacting (Z)-ethyl 2-chloro-2-(2-(4-methoxyphenyl)hydrazono)acetate of Formula (V) with 3-morpholino-1-(4-nitrophenyl)-5,6-dihydropyridin-2(1H)-one of Formula (VI) in one or more solvents in the presence of a base to obtain a compound of Formula (IV); 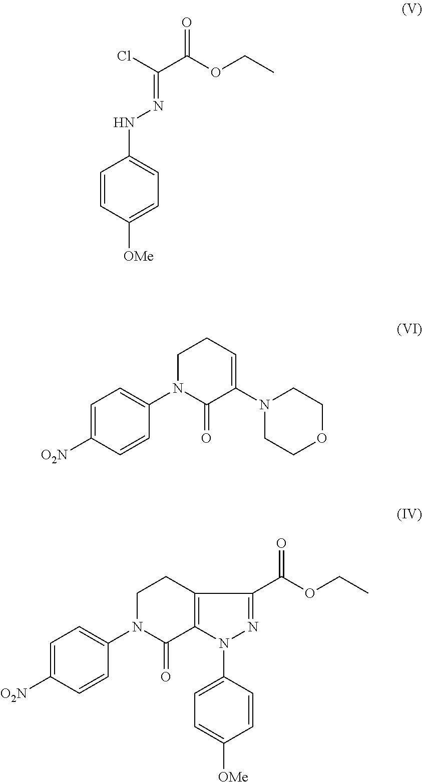 [SMILES: CCOC(=O)/C(Cl)=N/Nc1ccc(OC)cc1 ; O=C1C(N2CCOCC2)=CCCN1c1ccc([N+](=O)[O-])cc1 ; CCOC(=O)c1nn(-c2ccc(OC)cc2)c2c1CCN(c1ccc([N+](=O)[O-])cc1)C2=O]
(b) reducing the compound of Formula (IV) with a reducing agent to obtain a compound of Formula (III); 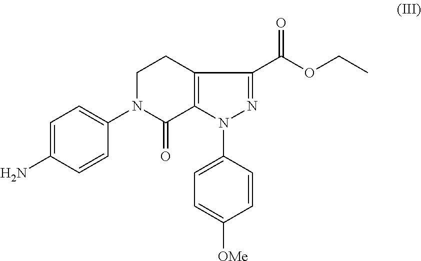 [SMILES: CCOC(=O)c1nn(-c2ccc(OC)cc2)c2c1CCN(c1ccc(N)cc1)C2=O]
(c) amidating the compound of Formula (III) with an amidating source in one or more solvents to obtain a compound of Formula (II); 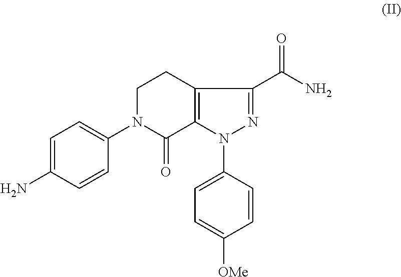 [SMILES: COc1ccc(-n2nc(C(N)=O)c3c2C(=O)N(c2ccc(N)cc2)CC3)cc1]
(d) reacting the compound of Formula (II) with 5-chlorovalaroyl chloride in the presence of a base to obtain a compound of Formula (IIB) as a crystalline solid; 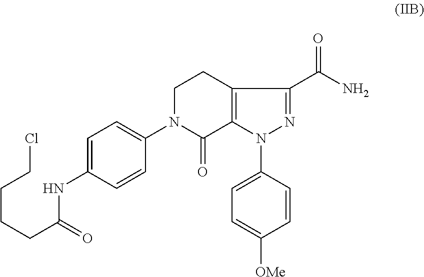 [SMILES: COc1ccc(-n2nc(C(N)=O)c3c2C(=O)N(c2ccc(NC(=O)CCCCCl)cc2)CC3)cc1]
(e) cyclizing the compound of Formula (IIB) in the presence of a base in one or more solvents to obtain the apixaban; and
(f) optionally, converting the apixaban obtained to an amorphous form.

28. The process according to claim 27, wherein the solvents in step (a) are selected from at least one member of the group consisting of methanol, ethanol, isopropanol, n-butanol, ethyl acetate, isopropyl acetate, butyl acetate, acetone, methylethyl ketone, methylisobutyl ketone, acetonitrile, dimethylformamide, dimethylacetamide, dimethylsulfoxide, N-methyl pyrrolidone, tetrahydrofuran, 2-methyl tetrahydrofuran, toluene, xylene, methylene dichloride, ethylene dichloride and mixtures thereof.

29. The process according to claim 27, wherein the base in step (a) is selected from at least one member of the group consisting of sodium hydroxide, potassium hydroxide, lithium hydroxide, sodium carbonate, potassium carbonate, lithium carbonate, sodium bicarbonate, potassium bicarbonate, sodium hydride, potassium hydroxide, sodium methoxide, sodium ethoxide, potassium tert-butoxide, ammonia, TEA, DIPA, DEA, DIPEA, DBU, DABCO, and DBN.

30. The process according to claim 27, wherein the reducing agent in step (b) comprises one or more of Raney Nickel, Pd/C, Pt/C, Platinum oxide, Fe—HCl, Fe—NH4C1, Sn—HCl, and Na₂Sx.

31. The process according to claim 27, wherein the amidating source in step (c) comprises one or more of formamide selected from N-ethylformamide, N-methylformamide, and formamide; or ammonia.

32. The process according to claim 27, wherein the solvents in step (c) comprise one or more of (a) alcohols selected from methanol, ethanol, isopropanol, or butanol; (b) ketones selected from acetone, methyl ethyl ketone, or methyl isobutyl ketone; or (c) esters selected from ethyl acetate, isopropyl acetate, or butyl acetate.

33. The process according to claim 27, wherein the base in step (d) is selected from at least one member of the group consisting of sodium hydroxide, potassium hydroxide, lithium hydroxide, sodium carbonate, potassium carbonate, lithium carbonate, sodium bicarbonate, potassium bicarbonate, sodium hydride, potassium hydroxide, sodium methoxide, sodium ethoxide, potassium tert-butoxide, ammonia, TEA, DIPA, DEA, DIPEA, DBU, DABCO, and DBN.

34. The process according to claim 27, wherein the crystalline compound of Formula (IIB) is crystalline characterized by having x-ray powder diffraction pattern comprising peaks expressed in degrees 2θ (±0.2° 2θ) at 7.9°, 10.9°, 15.8°, 16.2°, 19.6°, 21.8°, and 28.9° 2θ.

35. The process according to claim 27, wherein the compound of Formula (IV) is crystalline characterized by having x-ray powder diffraction pattern comprising peaks expressed in degrees 2θ (±0.2° 2θ) at 3.8°, 7.5°, 13.5°, 18.6°, 19.8°, 21.7°, 23.8°, and 25.5° 2θ.

36. The process according to claim 27, wherein the compound of Formula (III) is substantially amorphous having x-ray powder diffraction pattern comprising peaks expressed in degrees 2θ (±0.2° 2θ) at 4.8°, 9.4°, and 24.5° 2θ.

37. The process according to claim 27, wherein the compound of Formula (II) is crystalline characterized by having x-ray powder diffraction pattern comprising peaks expressed in degrees 2θ (±0.2° 2θ) at 18.4°, 21.2°, 22.4°, and 23.6° 2θ.

38. A process for preparing apixaban, the process comprising:
(a) reacting (Z)-ethyl 2-chloro-2-(2-(4-methoxyphenyl)hydrazono)acetate of Formula (V) with 3-morpholino-1-(4-nitrophenyl)-5,6-dihydropyridin-2(1H)-one of Formula (VI) in one or more solvents in the presence of a base to obtain a compound of Formula (IV); 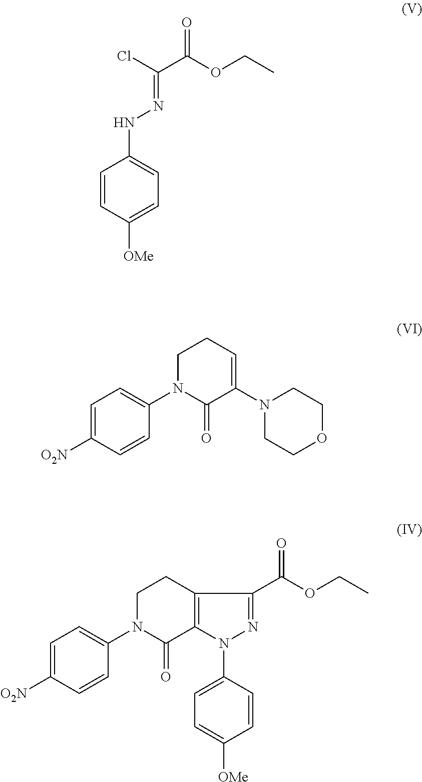 [SMILES: CCOC(=O)/C(Cl)=N/Nc1ccc(OC)cc1 ; O=C1C(N2CCOCC2)=CCCN1c1ccc([N+](=O)[O-])cc1 ; CCOC(=O)c1nn(-c2ccc(OC)cc2)c2c1CCN(c1ccc([N+](=O)[O-])cc1)C2=O]
(b) amidating the compound of Formula (IV) with an amidating source in one or more solvents to obtain a compound of Formula (IVA); 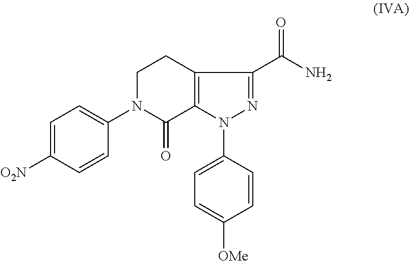 [SMILES: COc1ccc(-n2nc(C(N)=O)c3c2C(=O)N(c2ccc([N+](=O)[O-])cc2)CC3)cc1]
(c) reducing the compound (IVA) with a reducing agent to obtain a compound of Formula (II); 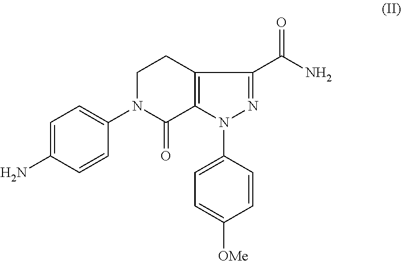 [SMILES: COc1ccc(-n2nc(C(N)=O)c3c2C(=O)N(c2ccc(N)cc2)CC3)cc1]
(d) reacting the compound of Formula (II) with 5-chlorovalaroyl chloride in the presence of a base to obtain a compound of Formula (IIB) as crystalline solid; 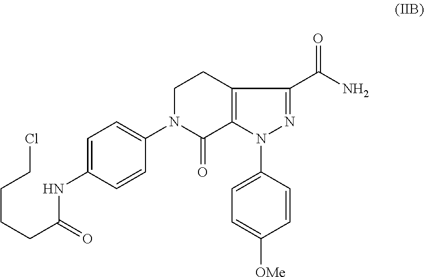 [SMILES: COc1ccc(-n2nc(C(N)=O)c3c2C(=O)N(c2ccc(NC(=O)CCCCCl)cc2)CC3)cc1]
(e) cyclizing the compound of Formula (IIB) in the presence of a base in one or more solvents to obtain the apixaban; and
(f) optionally, converting the apixaban obtained to an amorphous form.

39. The process according to claim 38, wherein the compound of Formula (IVA) is crystalline characterized by having x-ray powder diffraction pattern comprising peaks expressed in degrees 2θ (±0.2° 2θ) at 10.8°, 15.5°, 18.6°, 20.1°, 22.6°, 24.0°, and 27.4° 2θ.

40. A process for preparing apixaban, the process comprising:
(a) reacting a compound of Formula (III) with an amidating source to obtain a compound of Formula (II); 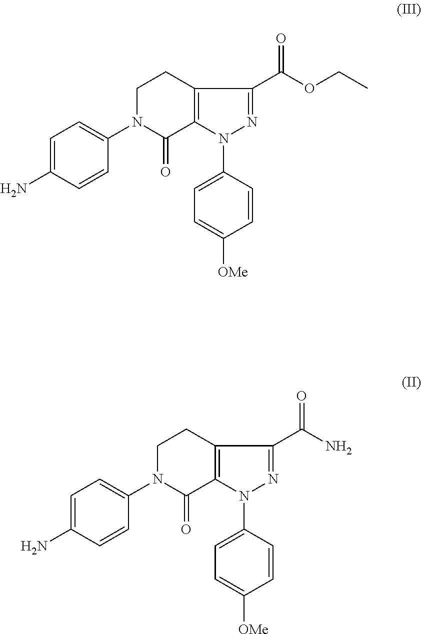 [SMILES: CCOC(=O)c1nn(-c2ccc(OC)cc2)c2c1CCN(c1ccc(N)cc1)C2=O ; COc1ccc(-n2nc(C(N)=O)c3c2C(=O)N(c2ccc(N)cc2)CC3)cc1]
(b) reacting the compound of Formula (II) with 5-chlorovalaroyl chloride in the presence of a base to obtain a compound of Formula (IIB) as crystalline solid; and 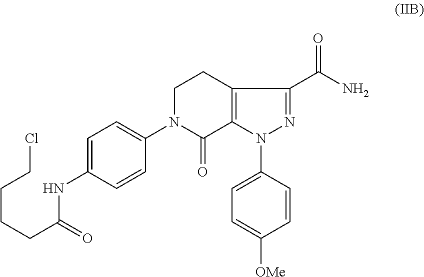 [SMILES: COc1ccc(-n2nc(C(N)=O)c3c2C(=O)N(c2ccc(NC(=O)CCCCCl)cc2)CC3)cc1]
(c) cyclizing the compound of Formula (IIB) to obtain the apixaban.

41. An isolated intermediate of apixaban comprising compounds of Formula (IIA), (IIB) and (IVA).

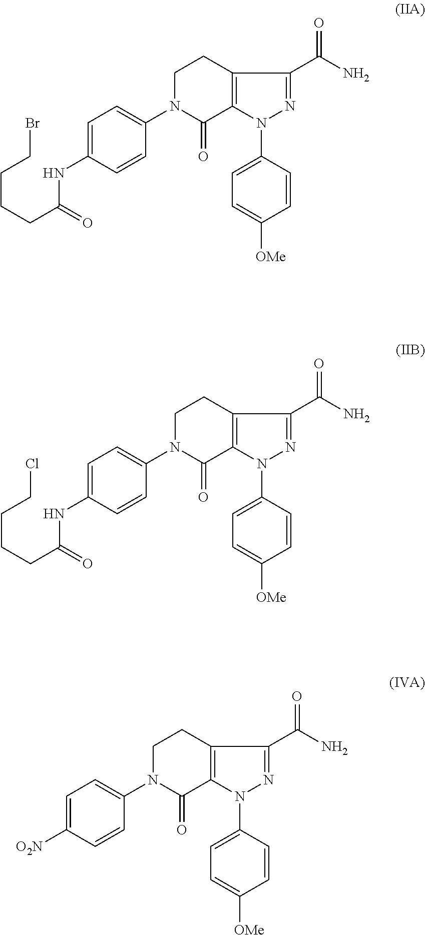 [SMILES: COc1ccc(-n2nc(C(N)=O)c3c2C(=O)N(c2ccc(NC(=O)CCCCBr)cc2)CC3)cc1 ; COc1ccc(-n2nc(C(N)=O)c3c2C(=O)N(c2ccc(NC(=O)CCCCCl)cc2)CC3)cc1 ; COc1ccc(-n2nc(C(N)=O)c3c2C(=O)N(c2ccc([N+](=O)[O-])cc2)CC3)cc1]

42. A crystalline form of apixaban characterized by having x-ray powder diffraction pattern comprising peaks expressed in degrees 2θ (±0.2° 2θ) at 5.9°, 6.9°, 13.4°, 14.9°, 16.0°, 17.3°, 21.4°, 22.5°, 24.2°, and 25.8°±0.2 2θ.

43. The crystalline form of apixaban according to claim 16 is further characterized by having x-ray powder diffraction pattern comprising peaks expressed in degrees 2θ (±0.2° 2θ) at 5.9°, 6.9°, 12.6°, 13.4°, 14.9°, 15.4°, 16.0°, 17.3°, 17.9°, 19.0°, 19.7°, 20.3°, 21.0°, 21.4°, 22.5°, 24.2°, 25.8°, 26.5°, 27.0°, 29.7°, 30.2° and 30.9°±0.2 2θ; having x-ray powder diffraction pattern substantially the same as that shown in FIG. 1; a differential scanning calorimetry having endothermic peak at about 103±5° C. and at about 151±5° C.; and a differential scanning calorimetry substantially the same as that shown in FIG. 2.

44. The crystalline apixaban according to claim 42 having purity of at least about 99% by area percentage of HPLC and a particle size distribution having (D10) of about 50 μm or less, (D50) of about 100 μm or less and (D90) of about 150 μm or less.

45. A pharmaceutical composition comprising crystalline apixaban according to claim 42 and one or more pharmaceutically acceptable excipients, diluents and carriers.

46. A pharmaceutical composition comprising an amorphous apixaban according to claim 27 and one or more of pharmaceutically acceptable carriers, excipients or diluents.
